(19) United States
(12) Patent Application Publication
(10) Pub. No.: US 2020/0140826 A1
(43) Pub. Date: May 7, 2020

(54) Maintenance and Expansion of Pancreatic Progenitor Cells

(71) Applicant: AGENCY FOR SCIENCE, TECHNOLOGY AND RESEARCH, Singapore (SG)

(72) Inventors: Jamie TROTT, Singapore (SG); Norris Ray DUNN, Singapore (SG)

(73) Assignee: Agency for Science, Research and Technology, Singapore (SG)

(21) Appl. No.: 16/478,307

(22) PCT Filed: Jan. 17, 2018
(86) PCT No.: PCT/SG2018/050029

§ 371 Date: Jul. 16, 2019

(30) Foreign Application Priority Data
Jan. 17, 2017 (SG) 10201700390Q

(51) Int. Cl. C12N 5/071

(52) U.S. Cl. CPC C12N 5/0678; C12N 2501/999; C12N 2501/39; C12N 2500/25; C12N 2501/01; C12N 2501/90; C12N 2501/155; C12N 2506/45; C12N 2501/11; C12N 2501/119; C12N 2506/02; C12N 2501/385; C12N 2501/117; C12N 2513/00; C12N 2501/16; C12N 2533/54; C12N 2501/727; C12N 2501/15; C12N 2502/1323; C12N 2533/90

(57) ABSTRACT

The present invention relates to a method of culturing a pancreatic progenitor cell. The method comprises contacting the cell with epidermal growth factor (EGF), retinoic acid (RA) and an inhibitor of transforming growth factor-β (TGF-β) and 3T3-J2 feeder cells. A cell produced by the method of the invention and a kit when used in the method are also provided.

### CROSS-REFERENCE TO RELATED APPLICATIONS

This application claims the benefit of priority of Singapore application No. 10201700390Q, filed 17 Jan.2017, the contents of it being hereby incorporated by reference in its entirety for all purposes.

### FIELD OF THE INVENTION

The invention is in the field of biotechnology. In particular, the present invention relates to methods for culturing pancreatic progenitor cells. The present invention further relates to culture mediums and kits for use in performing the methods as described herein.

### BACKGROUND OF THE INVENTION

The adult pancreas comprises three major lineages: endocrine, acinar, and ductal. The endocrine compartment resides in the islets of Langerhans and consists of cells that secrete hormones required for the maintenance of euglycemia, including a cells that secrete glucagon and β cells that secrete insulin and whose failure leads to diabetes. Acinar cells produce digestive enzymes and, together with duct cells, form the exocrine pancreas. Development of the human pancreas begins with the emergence of the dorsal and ventral pancreatic buds from the posterior foregut at Carnegie stage (CS) 12. These rudimentary structures consist of multipotent pancreatic progenitors that proliferate extensively and undergo branching morphogenesis before fusing to form the pancreatic anlage. Each of the three major pancreatic lineages is derived from these progenitor cells following a series of cell-fate decisions and morphological changes.

A series of genetic studies in mice led to the identification of numerous signaling pathways that regulate pancreatic development, thereby inspiring the development of protocols for the generation of pancreatic progenitors and subsequently b-like cells from human pluripotent stem cells. The ultimate goal of these studies is to generate functional β cells capable of maintaining euglycemia and alleviating diabetes. However, these protocols are technically challenging and expensive to conduct, often resulting in low differentiation efficiencies, partly due to the variability inherent in long, multi-step differentiation protocols that seek to recapitulate the entire developmental history of a β cell. These issues are exacerbated when such protocols are applied to genetically diverse embryonic stem cells (ESCs) and induced pluripotent stem cells (iPSCs). Accordingly, there is a need for the development of alternatives to pluripotent cells as a source for pancreatic cells that overcomes, or at least ameliorates, one or more of the disadvantages described above. There is also a need for the development of methods and culture conditions to support long-term self-renewal of these alternatives.

### SUMMARY

In one aspect, there is provided a method of culturing a pancreatic progenitor cell comprising contacting said cell with: a. epidermal growth factor (EGF); b. retinoic acid (RA); c. an inhibitor of transforming growth factor-β (TGF-β) signaling; and d. 3T3-J2 fibroblast feeder cells.

In one aspect, there is provided a cell produced according to the method of as described herein.

In one aspect, there is provided a kit when used in the method as described herein, comprising one or more containers of cell culture medium, together with instructions for use.

### DEFINITIONS

As used herein, the term “progenitor cell” refers to cells that have greater developmental potential, i.e., a cellular phenotype that is more primitive (e.g., is at an earlier step along a developmental pathway or progression) relative to a cell which it can give rise to by differentiation. Often, progenitor cells have significant or very high proliferative potential. Progenitor cells can give rise to multiple distinct cells having lower developmental potential, i.e., differentiated cell types, or to a single differentiated cell type, depending on the developmental pathway and on the environment in which the cells develop and differentiate. The term “pancreatic progenitor cell” refers to a cell that can give rise to multiple distinct cells of the pancreatic lineage. It is to be understood that a pancreatic progenitor cell is developmentally more proximal to specialized cells of the pancreas as compared to pluripotent stem cells. It will generally be understood that pancreatic progenitors may be derived using a variety of methods known in the art. In one example, pancreatic progenitors may be generated according to the method described in the Experimental Procedures section.

As used herein, “3T3-J2” in the context of feeder cells refers to the mouse embryonic fibroblast cell line, derived as described in Rheinwald J G, Green H. Serial cultivation of strains of human epidermal keratinocytes: the formation of keratinizing colonies from single cells. Cell. 1975 November;6(3):331-43 and Allen-Hoffmann B L, Rheinwald J G. Polycyclic aromatic hydrocarbon mutagenesis of human epidermal keratinocytes in culture.Proc Natl Acad Sci USA. 1984 December;81(24):7802-6.

As used herein, the term “inhibitor” in the context of signaling refers to a molecule or compound that interferes with the activity of a signaling pathway. An inhibitor may interfere with one or more members of a signaling pathway, its receptors or downstream effectors. An inhibitor may bind to one or more members of a signaling pathway, receptors or downstream effectors to inhibit biological function.

As used herein the phrase “culture medium” refers to a liquid substance used to support the growth of cells. The culture medium used by the invention according to some embodiments can be a liquid-based medium, for example water, which may comprise a combination of substances such as salts, nutrients, minerals, vitamins, amino acids, nucleic acids, proteins such as cytokines, growth factors and hormones.

As used herein, the term “embryonic stem cell” refers to naturally occurring pluripotent stem cells of the inner cell mass of the embryonic blastocyst. Such cells can similarly be obtained from the inner cell mass of blastocysts derived from somatic cell nuclear transfer. Embryonic stem cells are pluripotent and give rise during development to all derivatives of the three primary germ layers: ectoderm, endoderm and mesoderm. In other words, they can develop into each of the more than 200 cell types of the adult body when given sufficient and necessary stimulation for a specific cell type. They do not contribute to the extra-embryonic membranes or the placenta, i.e., are not totipotent.

As used herein, the term “induced pluripotent stem cells” or, iPSCs, means that the stem cells are produced from differentiated adult cells that have been induced or changed, i.e., reprogrammed into cells capable of differentiating into tissues of all three germ or dermal layers: mesoderm, endoderm, and ectoderm. The iPSCs produced do not refer to cells as they are found in nature.

As used herein, the term “differentiation” is the process by which an unspecialized (“uncommitted”) or less specialized cell acquires the features of a specialized cell such as, for example, a nerve cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. De-differentiation refers to the process by which a cell reverts to a less specialized (or committed) position within the lineage of a cell. As used herein, the lineage of a cell defines the heredity of the cell, i.e., which cells it came from and what cells it can give rise to. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Before the present inventions are described, it is to be understood that this invention is not limited to particular embodiments described, as such may vary. It is also to be understood that the terminology used herein is for purposes of describing particular embodiments only, and is not intended to be limiting, since the scope of the present invention will be limited only in the appended claims.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

The present disclosure and embodiments relate to methods of culturing a pancreatic progenitor cell that support long-term self-renewal. In particular, the present invention provides the knowledge of specific combinations of factors to maintain and expand pancreatic progenitors in culture.

Accordingly, the present invention provides a method of culturing a pancreatic progenitor cell comprising contacting said cell with:
a. epidermal growth factor (EGF); b. retinoic acid (RA); c. an inhibitor of transforming growth factor-β (TGF-β) signaling; and d. 3T3-J2 fibroblast feeder cells.

In one embodiment, the inhibitor of transforming growth factor-β (TGF-β) signaling may be an inhibitor of activin receptor-like kinase (ALK). It will generally be understood that there are seven identified ALKs. The inhibitor of ALK may inhibit one or more of ALK1 to ALK7. Advantageously, the inhibitor or ALK may inhibit one or more of ALK4, ALK5 or ALK7.

In one embodiment, the inhibitor of ALK may be a small molecule. In a preferred embodiment, the inhibitor of ALK may be SB431542.

In one embodiment, the pancreatic progenitor cell may be further contacted with one or more cell culture supplements. Cell culture supplements may be used to substitute for serum in cell culture media and improve cell viability and growth in culture. In a preferred embodiment, the pancreatic progenitor is further contacted with B27 supplement. It will generally be understood that other cell culture supplements or serum substitutes may also be used. Examples of cell culture supplements include but are not limited to 2-mercaptoethanol, amino acid solution, bovine serum albumin, cholesterol supplements, CHO supplement, glutamine, GlutaMax, primary cell supplements, HAT supplement, HT supplement, insulin, lipid supplement, MEM vitamin solution, pluronic F68, serum replacement, sodium pyruvate, stem cell supplements, transferrin, yeast solution, ITS-X supplement, N2 supplement and G5 supplement.

In one embodiment, the pancreatic progenitor cell may be further contacted with an inhibitor of Notch signaling. In a preferred embodiment, the inhibitor of Notch signaling may be a γ-secretase inhibitor. Examples of γ-secretase inhibitors include but are not limited to DAPT, RO4929097, Semagacestat, Compound E, gamma-Secretase Inhibitor III, (R)-Flurbiprofen, gamma-Secretase Inhibitor I, BMS-708163, BMS 299897, gamma-Secretase Inhibitor XI, JLK 6, Compound W, MK-0752, Dibenzazepine, LY411575, PF-03084014, L-685,458, gamma-Secretase Inhibitor VII, Compound 34, gamma-Secretase Inhibitor XVI.

In a further preferred embodiment, the y-secretase inhibitor may be DAPT.

In one embodiment, the pancreatic progenitor cell may be further contacted with dexamethasone, fibroblast growth factor 10 (FGF10), N2 supplement or combinations thereof.

Epidermal growth factor (EGF) may be used in an amount of from about 1 ng/mL to about 200 ng/mL, or from about 5 ng/mL to about 195 ng/mL, or from about 10 ng/mL to about 190 ng/mL, or from about 15 ng/mL to about 185 ng/mL, or from about 20 ng/mL to about 180 ng/mL, or from about 25 ng/mL to about 175 ng/mL, or from about 30 ng/mL to about 170 ng/mL, or from about 35 ng/mL to about 165 ng/mL, or from 40 ng/mL to about 160 ng/mL, or from about 45 ng/mL to about 155 ng/mL, or from about 50 ng/mL to about 150 ng/mL, or from about 55 ng/mL to about 145 ng/mL, or from about 60 ng/mL to about 140 ng/mL or from about 65 ng/mL to about 135 ng/mL, or from about 70 ng/mL to about 130 ng/mL, or from about 75 ng/mL to about 125 ng/mL, or from about 80 ng/mL to about 120 ng/mL or from about 85 ng/mL to about 115 ng/mL, or from about 90 ng/mL to about 110 ng/mL, or from about 95 ng/mL to about 105 ng/mL, or from about 95 ng/mL to about 100 ng/mL.

In a preferred embodiment, EGF may be used in an amount of about 50 ng/mL.

Fibroblast growth factor 10 (FGF10) may be used in an amount of from about 1 ng/mL to about 200 ng/mL, or from about 5 ng/mL to about 195 ng/mL, or from about 10 ng/mL to about 190 ng/mL, or from about 15 ng/mL to about 185 ng/mL, or from about 20 ng/mL to about 180 ng/mL, or from about 25 ng/mL to about 175 ng/mL, or from about 30 ng/mL to about 170 ng/mL, or from about 35 ng/mL to about 165 ng/mL, or from 40 ng/mL to about 160 ng/mL, or from about 45 ng/mL to about 155 ng/mL, or from about 50 ng/mL to about 150 ng/mL, or from about 55 ng/mL to about 145 ng/mL, or from about 60 ng/mL to about 140 ng/mL or from about 65 ng/mL to about 135 ng/mL, or from about 70 ng/mL to about 130 ng/mL, or from about 75 ng/mL to about 125 ng/mL, or from about 80 ng/mL to about 120 ng/mL or from about 85 ng/mL to about 115 ng/mL, or from about 90 ng/mL to about 110 ng/mL, or from about 95 ng/mL to about 105 ng/mL, or from about 95 ng/mL to about 100 ng/mL.

In a preferred embodiment, FGF10 may be used in an amount of about 50 ng/mL.

Retinoic acid (RA) may be used in an amount of from about 100 nM to about 10 μM,or from about 200 nM to about 9 μM,or from about 300 nM to about 8 μM,or from about 400 nM to about 7 μM,or from about 500 nM to about 6 μM,or from 600 nM to about 5 μM, or from about 700 nM to about 4 μM,or from about 700 nM to about 3 μM,or from about 800 nM to about 2 μM,or from about 900 nM to about 1 μM.

In a preferred embodiment, RA may be used in an amount of about 3 μM.

Dexamethasone may be used in an amount of from about 1 nM to about 100 nM, or from about 5 nM to about 95 nM, or from about 10 nM to about 90 nM, or from about 15 nM to about 85 nM, or from about 20 nM to about 80 nM, or from about 25 nM to about 75 nM, or from about 30 nM to about 70 nM, or from about 35 nM to about 65 nM, or from 40 nM to about 60 nM, or from about 45 nM to about 55 nM.

In a preferred embodiment, dexamethasone may be used in an amount of about 30 nM.

DAPT may be used in an amount of from about 100 nM to about 10 μM,or from about 200 nM to about 9 μM,or from about 300 nM to about 8 μM,or from about 400 nM to about 7 μM,or from about 500 nM to about 6 μM,or from 600 nM to about 5 μM,or from about 700 nM to about 4 μM,or from about 700 nM to about 3 μM,or from about 800 nM to about 2 μM,or from about 900 nM to about 1 μM.

In a preferred embodiment, DAPT may be used in an amount of about 1 μM.

SB431542 may be used in an amount of from about 1 μM to about 100 μM,or from about 5 μM to about 95 μM,or from about 10 μM to about 90 μM,or from about 15 μM to about 85 μM,or from about 20 μM to about 80 μM,or from about 25 μM to about 75 μM, or from about 30 μM to about 70 μM,or from about 35 μM to about 65 μM,or from about 40 μM to about 60 μM,or from about 45 μM to about 55 μM,or about 50 μM.

In a preferred embodiment, SB431542 may be used in an amount of about 10 μM.

With respect to cell culture supplements, it will be generally understood that cell culture supplements may be obtained in concentrated form (e.g. 10×, 50× or 100×). Accordingly, it will be understood that cell culture supplements may be diluted and used at a final concentration of about 1×, 2×, 3×, 4× or 5×.

In a preferred embodiment, N27 and B27 may be used at a final concentration of 1×.

Accordingly, in one embodiment, the pancreatic progenitor cell may be contacted with about 1 ng/ml to about 100 ng/ml of EGF, about 100 nM to about 10 μM of RA, and about 1 μM to about 100 μM of SB431542.

In one embodiment, the pancreatic progenitor cell may be contacted with about 1 ng/ml to about 100 ng/ml of EGF, about 1 ng/ml to about 100 ng/ml of FGF10, about 100 nM to about 10μM of RA, about 1 nM to about 100 nM of dexamethasone, about 100 nM to about 10 μM DAPT, about 1 μM to about 100 μM of SB431542, about 1× B27 supplement; and about 1× N2 supplement.

In a preferred embodiment, the pancreatic progenitor cell is contacted with about 50 ng/mL EGF, about 50 ng/ml FGF10, about 3 μM RA, about 30 nM dexamethasone, about 1 μM DAPT, about 10 μM SB431542, about 1× B27 supplement, and about 1× N2 supplement.

In one embodiment, the pancreatic progenitor cell may be a pancreatic progenitor cell population. The pancreatic progenitor cell population may be substantially homogenous. Substantially homogenous as used herein means that the majority of cells in the population are pancreatic progenitor cells.

The pancreatic progenitor cell population may be at least 60%, at least 70%, at least 80%, at least 90%, at least 95%, at least 99% homogenous. In a preferred embodiment, the pancreatic progenitor cell population is more than 99% homogenous.

The method of the present invention allows long-term culture of the pancreatic progenitor cell. In one embodiment, the pancreatic progenitor cell may be cultured for at least 5 passages, at least 10 passages, at least 15 passages, at least 20 passages or at least 30 passages.

In one embodiment, the pancreatic progenitor cell may be derived from a stem cell. The stem cell may be an embryonic stem cell or an induced pluripotent stem cell.

In one embodiment, the pancreatic progenitor cell may express markers of the endoderm and pancreas lineages. In one embodiment, the pancreatic progenitor cell may express one or more of PDX1, SOX9, HNF6, FOXA2 and GATA6. In another embodiment, the pancreatic progenitor cell may not express SOX2.

In one embodiment, the method of culturing the pancreatic progenitor cell prevents differentiation of the pancreatic progenitor cell. In another embodiment, the method of culturing the pancreatic progenitor cell promotes proliferation of the pancreatic progenitor cell.

In another aspect of the invention, there is provided a cell produced by the method of the present invention.

In another aspect of the invention, there is provided a kit when used in the method of the present invention, comprising one or more containers of cell culture medium. The components of the cell culture medium may be provided in one or more containers individually or in combination. In one embodiment, the kit further comprises 3T3-J2 feeder cells.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification, and variation of the inventions embodied therein herein disclosed may be resorted to by those skilled in the art, and that such modifications and variations are considered to be within the scope of this invention.

The disclosure has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

### BRIEF DESCRIPTION OF THE DRAWINGS

The invention will be better understood with reference to the detailed description when considered in conjunction with the non-limiting examples and the accompanying drawings, in which:

FIG. 1 shows the generation of hiPSC lines from diabetic and healthy sibling fibroblasts. FIG. 1A shows the pedigree of a consanguineous Jordanian family with several diabetic siblings. All diabetic siblings developed the disease before 5 years of age. Skin biopsies taken from individuals AK5 and AK6 were used to generate fibroblasts from which hiPSC were derived. FIG. 1B shows the intracellular flow cytometric analysis of OCT4 expression and FIG. 10 shows immunostaining for established markers of pluripotency for hiPSC clones AK5-11, AK6-13 and AK6-8 (not shown). Scale bar, 100 μm.

FIG. 2 shows the directed differentiation of pancreatic progenitor cells and generation of cultured pancreatic progenitor (cPP) cells from diverse human pluripotent stem cell lines. FIG. 2A shows the time-course of pancreatic progenitor differentiation protocol. In these experiments, stage 1 was extended to last 3 days, rather than 2 as per the manufacturer's instructions, by repeating the final day's treatment. FIG. 2B shows the intracellular flow cytometric analysis of PDX1 and NKX6-1 at days 8, 10 and 15 of differentiation using hES3 INS-GFP reporter hESC and the in-house hiPSC lines AK5-11 and AK6-8. PDX1 is detected before NKX6-1 in all cases, although individual lines exhibit variable differentiation kinetics. Gates are based on cells stained with isotype control antibodies. FIG. 2C shows the percentage of PDX1+ and/or NKX6-1+ at day 15 of differentiation. Each circle represents one of 31 independent experiments encompassing 2 hESC lines and 6 hiPSC lines. The vertical black bar shows the median percentage of cells that are PDX1+ (95%), NKX6-1+ (80%) or PDX1+NKX6-1+ (80%). FIG. 2D shows gene expression measured by qRT-PCR using samples harvested from cPP cell lines at passage 6. The study analyzed cPP cells derived from the following pluripotent cell lines: H9 and HES3 hESC, and AK5-11, AK6-8 and AK6-13 hiPSC. Two independent pedigrees were derived from H9 and AK5-11 cell lines. Expression levels are shown normalized to those of H9 hESC and are plotted on a loge scale. Error bars represent the standard error of three technical replicates.

FIG. 3 shows the derivation of cPP Cell Lines from hESC and hiPSC. FIG. 3A shows pancreatic progenitors generated after 15 days of differentiation using the STEMdiff directed differentiation kit (PPd15 cells) were plated and expanded on a layer of 3T3-J2 feeder cells in medium supplemented with the indicated growth factors and signaling inhibitors. FIG. 3B shows the intracellular flow cytometric analysis for PDX1 and NKX6-1 at days 8,10, and 15 of differentiation using H9 hESCs. FIG. 3C shows phase-contrast images of cPP cells passaged as aggregates (left) and as single cells (right). Scale bar, 100 μm. FIG. D shows gene expression measured by qRT-PCR using samples harvested from PPd15 cells and cPP cells at early (6-8), middle (11-13), and late (14-18) passages. Cells were derived from both AK6-13 hiPSC and H9 hESC. Gene expression in definitive endoderm (H9 hESCs after 4 days STEMdiff differentiation) is shown for comparison. Values are plotted on a log₂ scale and error bars represent the SE of three technical replicates. ND, not detected. FIG. 3E shows immunofluorescence staining of cPP cells for key pancreatic transcription factors. Scale bar, 100 μm. FIG. 3F shows intracellular flow cytometric analysis of cPP cells for PDX1. Gray dots represent control cells stained with isotype control antibodies. Intracellular flow cytometric analysis of cPP cells for PDX1. Gray dots represent control cells stained with isotype control antibodies.

FIG. 4 shows that chromosome counting and M-FISH analysis reveals cPP Cells are genetically stable. FIG. 4A shows chromosome counting of cPP cells from diverse genetic backgrounds at different passage numbers. Values shown are the percentage of spreads with a given number of chromosomes, with the modal chromosome count for each cPP line highlighted. A modal (shared by >80% of cells) chromosome number of 46 is indicative of a normal karyotype and of karyotypic stability. Five out of six cPP cell lines analyzed exhibited a modal chromosome count of 46 after >6 passages, without evidence of fragments or dicentric chromosomes, and are considered karyotypically stable. In H9 pedigree #1, cells gradually acquired an additional isochromosome upon passaging. Traditional G-band karyotyping (data not shown) subsequently found this to be i(12) (p10)[20], an isochromosome commonly observed in hESC cultures. FIG. 4B shows multicolor fluorescence in situ hybridization (M-FISH) enables the detection of chromosomal structural abnormalities at significantly higher resolution than chromosome counting alone. M-FISH of passage 20 AK6-13 cPP cells failed to detect aneuploidy, translocations or deletions in 19/20 spreads analyzed. A representative image of a single chromosome spread is shown.

FIG. 5 shows transcriptome analysis of cPP cells by RNA-seq. FIG. 5A shows correlations between gene expression levels for cPP cells from three different genetic backgrounds (H9, AK6-13 and HES3) at early (6-8), mid (11-13) and late (18) passages. Log2-transformed gene counts measured by RNA-seq were plotted for each gene. Gene counts in cPP samples are compared to liver for comparison. The Spearman correlation coefficient for each pair of samples is shown on the corresponding plot. Heat colors denote the number of transcripts. Gene counts are strongly correlated between cPP samples regardless of genetic background or passage number, but not with liver. FIG. 5B shows the identification of specifically expressed genes in liver, lung and colon samples. Genes associated with early pancreatic development are not typically found to be specifically expressed by these tissues. FIG. 5C shows Z-score correlations for cPP, PPd15, CS16-18 PP and liver samples. Z-scores are strongly correlated between in vitro and in vivo pancreatic progenitor samples but not between these samples and liver.

FIG. 6 shows the transcriptome analysis of cPP Cells by RNA-Seq. FIG. 6A shows Hierarchical clustering of Euclidean distances between transcriptomes of diverse adult and embryonic tissues shows that in vitro and in vivo pancreatic progenitors exhibit similar patterns of gene expression. Log2-transformed gene count values were used to calculate Euclidian distances. For detailed information on the sources of data used here, see Table 1 FIG. 6B shows heatmaps of loge-transformed gene expression levels of key endodermal and pancreatic markers by in vitro and in vivo pancreatic progenitors. Levels in brain are shown for comparison. FIG. 6C shows genes specifically expressed by cPP, PPd15, and CS16-18 pancreatic progenitors. The coefficient of variance (CV) for each protein coding gene across the 25 tissues shown in FIG. 6A was plotted against the corresponding Z score. Specifically expressed genes are located in the upper right-hand quadrant (CV >1 and Z score >1) and include genes with well-characterized roles in early pancreatic development (labeled). The color scale denotes the number of genes. The Venn diagram shows overlap between genes specifically expressed by cPP, PPd15, and CS16-18 pancreatic progenitors. FIG. 6D shows biological process Gene Ontology (GO) terms associated with all genes specifically expressed by cPP cells (above) or genes specifically expressed by cPP cells but not PPd15 or CS16-18 pancreatic progenitors (below). Only GO terms associated with >5 genes and/or an adjusted p value <0.01 are shown. FIG. 6E shows the heatmap of expression levels of genes associated with the enriched GO terms mitotic recombination, DNA strand elongation, telomere maintenance, and DNA packaging. Levels are shown for individual cPP and PPd15 populations derived from three different genetic backgrounds (H9, AK6-13, and HES3) relative to the maximum detected value across the 25 different tissues shown in FIG. 6A. FIG. 6F shows the expression of selected telomerase pathway genes as measured by qRT-PCR in cPP and PPd15 cells. Error bars represent the SE of three technical replicates.

FIG. 7 shows that a layer of 3T3-feeder cells and exogenous signaling molecules are required for the maintenance and expansion of cPP cells. FIG. 7A shows phase-contrast images of H9 and AK6-13 cPP cells after 7 days culture in complete medium on 3T3-feeder cells plated at densities of 5×10⁴, 2.5×10⁴, and 1.25×10⁴ cells/cm². Scale bar, 100 μm. FIG. 7B shows gene expression measured by qRT-PCR for samples harvested from cultures in FIG. 7A for endocrine (NGN3 and NKX2-2), ductal (KRT19 and CA2), and acinar (CPA1 and AMY2B) marker genes. Error bars represent the SE of three technical replicates. FIG. 7C shows phase-contrast images of cPP cells cultured for 6 days in complete medium with individual components omitted. Scale bar, 100 μm. FIG. 7D shows PDX1 and SOX9 expression measured by qRT-PCR for samples harvested in (C). Error bars represent the SE of three technical replicates. FIG. 7E shows Microbioreactor array (MBA) screening of factors required to propagate PDX1+SOX9+ cPP cells. Effects of reducing or removing selected factors (EGF, RA, DAPT) from complete medium containing all factors at the following levels: EGF (50 ng/mL), RA (3 μM), DAPT (1 μM), SB431542 (10 μM), and FGF10 (50 ng/mL). Top panels: effects on total nuclei per chamber, and PDX1 and SOX9 mean nuclear intensity. Lower panels: effects on the total number of PDX1+SOX9+ cells per chamber and percentage of PDX1+SOX9+ cells. Data represent the mean of ten chambers within a column treated with the given condition ±the SE. FIG. 7F shows a heatmap of RNA-seq expression levels of components of signaling pathways that regulate cPP proliferation: EGF (EGFR), FGF10 (FGFR1-4, 6 and FGFRL2), RA (RARA, RARB, RARG, RXRA, RXRB, and RXRG), SB431542 (ACVR1B [ALK4], TGFBR1 [ALKS], and ACVR1C [ALK7]), and DAPT (NOTCH1-4 and its ligands DLL1,3,4 and JAG1,2). Levels are shown relative to those observed across all 25 tissues shown in FIG. 6A.

FIG. 8 shows Microbioreactor Array (MBA) screening of factors required to propagate cPP cells. FIG. 8A shows Phase contrast images of PDX1+SOX9+ cPP cells seeded into Matrigel-coated MBAs and allowed to attach for 20 h with periodic feeding. Each MBA device has 270 chambers arranged as shown in S4D. Scale bar, 100 μm. FIG. 8B shows the protocol used for MBA screening. FIG. 8C shows individual chambers of MBA device (270 culture chambers) stained with anti-PDX1 (green) and anti-SOX9 (red) antibodies. Hoechst 33342 (not shown) was used for nuclei identification. The chambers were selected to show the range of proliferation rates and protein expression observed across different signaling environments. Scale bar, 100 μm. FIG. 8D shows endpoint measurements for each chamber in the MBA. Schematic above shows compositions of media applied to each column of the MBA (EGF, ng/mL; RA, pM; DAPT, pM). Cell culture media flow was from top (Row 1) to bottom (Row 10) down a column, thereby concentrating autocrine factors towards the bottom of the column. Mean measurements for each column are given below. QCF: data flagged for quality control issue during image processing. Values were extracted from images such as those in S4C using an image segmentation algorithm as described previously.

FIG. 9 shows the testing of cPP Potency in vitro and in vivo. FIG. 9A shows feeder-depleted passage 15 H9 cPP cells were replated on Matrigel and exposed to the indicated factors that promote multilineage differentiation toward the endocrine, duct, and acinar lineages. FIG. 9B shows endocrine, exocrine, and ductal gene expression analysis in FIG. 9A after 3, 6, and 12 days. Values are shown relative to levels in undifferentiated cPP cells (day 0). Error bars represent the SE of three technical replicates. FIG. 9C shows directed differentiation of passage 10 AK6-13 cPP cells to insulin+ b-like cells using a modified version of Russ et al. (2015). FIG. 9D shows phase-contrast image of differentiating spheres undergoing branching morphogenesis after 4 days. Scale bar, 100 μm. FIG. 9E shows that intracellular flow cytometric analysis of day 4 cells shows approximately 70% reactivate NKX6-1 and maintain PDX1. FIG. 9F shows PDX1 and NKX6-1 immunostaining on day 4. Scale bar, 100 μm. FIG. 9G shows that on day 9, the majority of cells are NKX2-2+ with a proportion of these transiently NGN3+. Scale bar, 100 μm. FIG. 9H shows the phase contrast image of day 16 spheres. Scale bar, 100 μm. FIG. 9I shows that approximately 20% of cells are C-peptide+ on day 16. FIG. 9J shows that day 16 C-peptide+ cells do not coexpress glucagon. Scale bar, 100 μm. FIG. 9K shows gene expression measured by qRT-PCR of cPP cells on days 4, 9, and 16 harvested from the differentiation protocol in FIG. 9C. Levels are shown relative to those in undifferentiated cPP cells and human islets for comparison. Error bars represent the SE of three technical replicates. FIG. 9L shows immunostaining of transplanted cPP cells for markers of endocrine (C-peptide and glucagon), duct (keratin-19), and acinar (trypsin) lineages. Scale bar, 100 μm.

FIG. 10 shows the optimization of cPP beta cell differentiation. FIG. 10A shows the application of NKX6-1 induction step of published beta cell differentiation protocols to cPP cells. This study established 2D-monolayer, 3D-matrigel and 3D-suspension cultures in complete cPP media before exposing cells to growth factor regimes based on published beta cell differentiation protocols. Phase contrast images were taken at the end of each treatment. Scale bar, 100 μm. FIG. 10B shows gene expression measured by qRT-PCR using samples harvested in FIG. 10A. When cells were exposed to the Rezania and Pagliuca differentiation regimes using the 3D matrigel platform, sufficient material to carry out qRT-PCR analysis was unable to be recovered. Error bars represent the standard error of three technical replicates. FIG. 100 shows optimization of the NKX6-1 induction step of the Russ et al. differentiation regime. Differentiations were carried out using the 3D-suspension platform. The lengths of the two growth factor treatments were varied to maximize the percentage of cells that reactivate NKX6-1 expression. PDX1 and NKX6-1 were measured by intracellular flow cytometric analysis. Three independent experiments are shown for each condition. FIG. 10D shows the percentage PDX1+NKX6-1+ cells generated in FIG. 100.

### EXPERIMENTAL SECTION

Non-limiting examples of the invention and comparative examples will be further described in greater detail by reference to specific Examples, which should not be construed as in any way limiting the scope of the invention.

Experimental Procedures

Human Pluripotent Stem Cell Culture and Differentiation

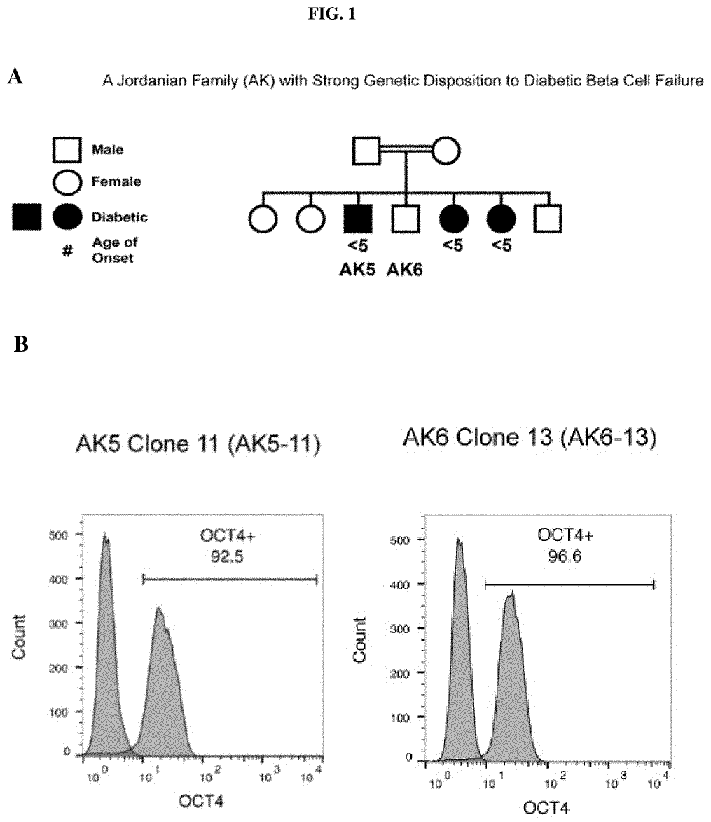

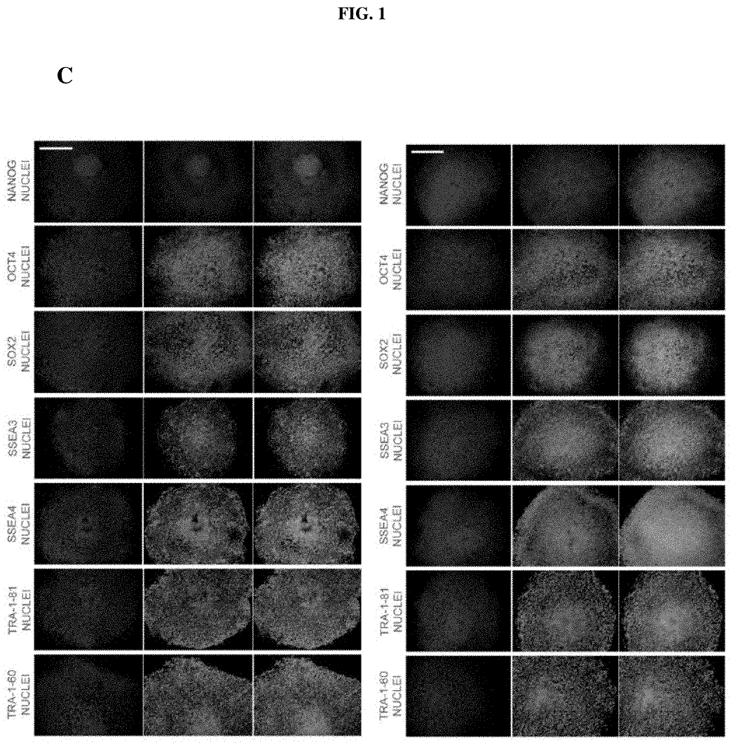

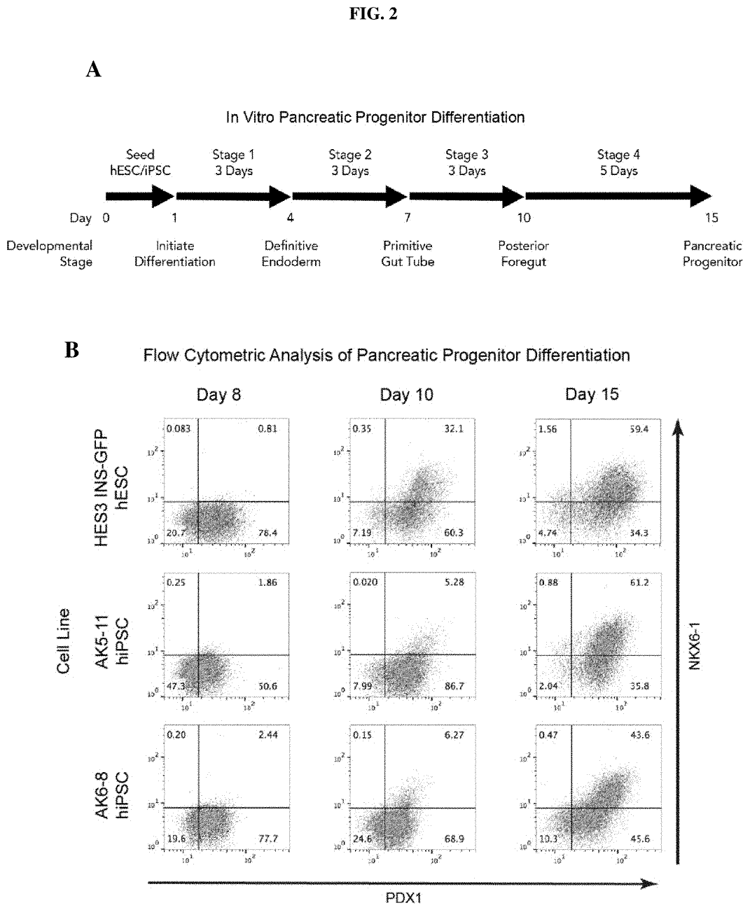

The following hESC lines were used in this study: H9 (WA09) were purchased from WiCell, HES3 (ES03) were provided by ES Cell International Pte. Ltd., and the HES-3 INSGFP/w reporter line was a gift from the Stanley lab (Micallef et al., 2011). The hiPSC lines used in this study were derived inhouse from human fibroblasts and are designated AK5-11, AK6-8 and AK6-13 (FIG. 1). Pluripotent stem cells were maintained on tissue culture plastic coated with Matrigel in mTeSR1 medium as described previously, and differentiated into pancreatic progenitors using the STEMdiff Pancreatic Progenitor kit (STEMCELLTechnologies, 05120) according to the manufacturer's instructions with the following modifications: (1) cells were initially seeded into 12-well plates (Corning, 353043) at a density of 106 cells/well, and (2) stage 1 was extended to 3 days by repeating the final day's treatment. All tissue culture was carried out in 5% CO2 at 37° C.

Generation of hiPSC

Fibroblasts were obtained by punch skin biopsy and reprogrammed to generate hiPSC. Fibroblasts were reprogrammed using the CytoTune™-iPS 2.0 Sendai Reprogramming Kit (Thermo Fisher Scientific, A16517) in accordance with the manufacturer's instructions. Cells were passaged and plated onto irradiated mouse embryonic feeders 7 days after viral transfection. Thereafter, hiPSC colonies were picked between days 17-28 and maintained in DMEM/F12 (Sigma, D6421) supplemented with 20% Knock Out Serum Replacement (Thermo Fisher Scientific, 10828-028), 0.1 mM 2-mercaptoethanol (Thermo Fisher Scientific, 21985-023), 2 mM L-glutamine (Thermo Fisher Scientific, 25030), 0.2 mM NEAA (Thermo Fisher Scientific, 11140-050) and 5 ng/mL bFGF (Peprotech, 100-18B). Staining with the following antibodies was used to confirm pluripotency (FIG. 1): NANOG (R&D Systems, AF1997, 1:200), OCT4 (Santa Cruz, 111351, 1:200), SOX2 (R&D Systems MAB2018, 1:200), SSEA3 (Millipore, MAB4303, 1:50), SSEA4 (Millipore, MAB4304, 1:200), TRA-1-81 (Millipore, MAB4381, 1:200), TRA-1-60 (Millipore, MAB4360, 1:200). Primary antibodies were recognized by Alexa-fluorophore conjugated secondary antibodies raised in Donkey (Thermo Fisher Scientific, 1:500). The study protocol was approved by the National University of Singapore Institutional Review Board (NUS IRB 10-051). The study was conducted in accordance with the Declaration of Helsinki and written informed consent was obtained from the participants.

Passaging and Maintenance of Cultured Pancreatic Progenitor (cPP) Cells

Gentle cell dissociation reagent (STEMCELL Technologies, 07174) was used to passage cPP cells as aggregates that were then seeded at a 1:6 split ratio onto a layer of 3T3-J2 feeders (0.5×10⁶ to 1×10⁶ cells/cm²) in medium composed of advanced DMEM/F12 (Thermo Fisher Scientific, 21634010), 2 mM L-glutamine (Thermo Fisher Scientific, 25030), 100 U/mL penicillin/streptomycin (Thermo Fisher Scientific, 15140122), 1× N2 supplement (Thermo Fisher Scientific, 17502-048), 1× B27 supplement (Thermo Fisher Scientific, 17504-044), 30 nM dexamethasone (STEMCELL Technologies, 72092), 50 ng/mL EGF (R&D Systems, 236-EG-200), 50 ng/mL FGF10 (Source Bioscience, ABC144), 3 μM RA (Sigma, R2625), 10 μM SB431542 (Calbiochem, 616464), and 1 μM DAPT (Sigma, D5942). If plating single cPP cells, complete medium was supplemented with 10 μM Y27632 for the first 48 hr (Sigma, Y0503). Medium was completely replenished every 2-3 days.

Expansion of 3T3-J2 feeders

3T3-J2 feeder cells (passage 9) were expanded on tissue culture plastic (coated with 0.1% gelatin (Sigma, G2625) for 30 min) in 3T3-J2 culture media and passaged as single cells by treating with 0.25% Trypsin for 5 min (Thermo Fisher Scientific, 25200056). 3T3-J2 culture media is composed of the following: DMEM high glucose (Thermo Fisher Scientific, 11960), 10% Fetal Bovine Serum (FBS, ES cell qualified, Thermo Fisher Scientific, 16141079), 2 mM L-glutamine (Thermo Fisher Scientific, 25030), and 100 U/mL penicillin/streptomycin (Thermo Fisher Scientific, 15140122). Feeder cells were mitotically inactivated by gamma irradiation (20 grays for 30 min) then frozen in culture media +DMSO. Individual batches of FBS are selected to enable 3T3-J2 cells to maintain cPP cultures, whilst 3T3-J2 cells are never cultured beyond passage 12 and should be seeded at 3.5-5×10³ cells/cm² and not allowed to exceed 1.3×10⁴ cells/cm².

Preparation of 3T3-J2 Feeder-Coated Culture Vessels

Thawed 3T3-J2 cells were seeded at 0.5-1×10⁶cells/cm² onto tissue culture plates coated with 0.1% gelatin (Sigma, G2625) for 30 min and maintained in 3T3-J2 culture media for up to 3 days until required. The optimal plating density must be determined empirically for each batch of feeders and is assessed based on the ability to maintain colony morphology without significantly hindering growth, since increasing feeder density improves colony morphology and blocks differentiation, but results in reduced proliferation rates. Tissue culture vessels containing feeders were washed once with DMEM to remove residual FBS prior to addition of cPP culture media.

### Metaphase Spread Preparation, Chromosome Counting and M-FISH Karyotyping

Cells grown to ˜75-80% confluency were treated with 100 ng/ml Colcemid solution (Gibco, 15212012) for 6 h, trypsinized and centrifuged at 1000 rpm for 10 min. Cell pellets were resuspended in 75 mM KCl and incubated for 15 min in a 37° C. waterbath. 1/10 volume of 3:1 methanol/acetic acid was added to cells followed by centrifugation at 1000 rpm for 15 min. Cells were then fixed by resuspension in 3:1 methanol/acetic acid solution, incubated for 30 min at room temperature, centrifuged at 1200 rpm for 5 min and finally washed once more with fixative. Cells were resuspended in a small volume of fixative, dropped onto clean glass slides and left to air dry. Multicolor FISH (MFISH) was performed according to manufacturer's instructions (MetaSystems). Automated acquisition of chromosome spreads was performed using Metafer imaging platform (MetaSystem). Ikaros and Fiji software were used to determine the chromosome number per spread and analyze M-FISH images.

### RNA-Seq Analysis of Gene Expression

RNA was isolated from samples harvested from cPP and PPd15 cultures using an RNeasy mini kit (QIAGEN, cat. no. 74104). Feeder removal microbeads (Miltenyi Biotec, 130-095-531) were used to deplete cPP cells of 3T3 feeders prior to RNA extraction. All RNA samples had an RNA integrity number >9. RNA-seq libraries were generated using the NEBNext Ultra RNA Library Prep Kit (NEB, E7530L) and sequenced on an Illumina HiSeq 2500 system generating single-end reads of 100 bp. Table 1 contains metadata for these and public datasets used for the RNA-seq gene expression analysis.

TABLE 1
Metadata used for RNA-seq gene expression analysis.

| | | | Uniquely | | |
|---|---|---|---|---|---|
| | | | aligned | Read | |
| | | Total | reads (% | length | |
| Sample | Stage | reads | total) | (bp) | Source of data |
| H9 cPP | Passage 8 | 44620737 | 0.902 | 100 | This study E-MTAB-5731 |
| Early | | | | | (ArrayExpress Archive) |
| H9 cPP | Passage 13 | 44869916 | 0.902 | 100 | This study E-MTAB-5731 |
| Mid | | | | | (ArrayExpress Archive) |
| H9 cPP | Passage 18 | 41431366 | 0.897 | 100 | This study E-MTAB-5731 |
| Late | | | | | (ArrayExpress Archive) |
| AK6-13 | Passage 6 | 45586537 | 0.903 | 100 | This study E-MTAB-5731 |
| cPP Early | | | | | (ArrayExpress Archive) |
| AK6-13 | Passage 11 | 41439771 | 0.888 | 100 | This study E-MTAB-5731 |
| cPP Mid | | | | | (ArrayExpress Archive) |
| HES3 cPP | Passage 8 | 37249318 | 0.900 | 100 | This study E-MTAB-5731 |
| Early | | | | | (ArrayExpress Archive) |
| H9 PPd15 | Day 15 | 45700168 | 0.891 | 100 | This study E-MTAB-5731 |
| Pancreatic | | | | | (ArrayExpress Archive) |
| Progenitors | | | | | |
| AK6-13 | Day 15 | 27282321 | 0.889 | 100 | This study E-MTAB-5731 |
| PPd15 | | | | | (ArrayExpress Archive) |
| Pancreatic | | | | | |
| Progenitors | | | | | |
| HES3 | Day 15 | 30324745 | 0.919 | 100 | This study E-MTAB-5731 |
| PPd15 | | | | | (ArrayExpress Archive) |
| Pancreatic | | | | | |
| Progenitors | | | | | |
| Cebola In | Day 12 | 42903206 | 0.784 | 90 | E-MTAB-3061 (Cebola et al |
| vitro | | | | | 2015, ArrayExpress Archive) |
| Pancreatic | | | | | |
| Progenitors | | | | | |
| Cebola | CS16-18 | 46455693 | 0.761 | 90 | E-MTAB-3061 (Cebola et al |
| CS16-18 | | | | | 2015, ArrayExpress Archive) |
| Pancreatic | | | | | |
| Bud | | | | | |
| Beta Cell | 59 years | 64513275 | 0.789 | 36 | E-MTAB-1294 (Moran et al |
| | | | | | 2012, ArrayExpress Archive) |
| Embryonic | day 91 | 62760353 | 0.668 | 36 | SRX214006 (SRA accession |
| heart | | | | | number) |
| Embryonic | day 91 | 42092063 | 0.644 | 36 | SRX343530 (SRA accession |
| muscle | | | | | number) |
| Embryonic | day 112 | 60726935 | 0.738 | 36 | SRX343522 (SRA accession |
| spleen | | | | | number) |
| Embryonic | day 115 | 76123335 | 0.706 | 36 | SRX343526 (SRA accession |
| thymus | | | | | number) |
| Embryonic | 140-231 days | 9896201 | 0.657 | 51 | SRX208133 (SRA accession |
| brain | | | | | number) |
| Adipose | 73 years | 76784649 | 0.790 | 50 | E-MTAB-513 (ArrayExpress |
| | | | | | Archive, Illumina Human Body |
| | | | | | Map 2 project) |
| Adrenal | 60 years | 75322220 | 0.811 | 50 | E-MTAB-513 (ArrayExpress |
| | | | | | Archive, Illumina Human Body |
| | | | | | Map 2 project) |
| Brain | 77 years | 68913126 | 0.856 | 50 | E-MTAB-513 (ArrayExpress |
| | | | | | Archive, Illumina Human Body |
| | | | | | Map 2 project) |
| Breast | 29 years | 76528738 | 0.802 | 50 | E-MTAB-513 (ArrayExpress |
| | | | | | Archive, Illumina Human Body |
| | | | | | Map 2 project) |
| Colon | 68 years | 81347600 | 0.812 | 50 | E-MTAB-513 (ArrayExpress |
| | | | | | Archive, Illumina Human Body |
| | | | | | Map 2 project) |
| Heart | 77 years | 79842823 | 0.819 | 50 | E-MTAB-513 (ArrayExpress |
| | | | | | Archive, Illumina Human Body |
| | | | | | Map 2 project) |
| Kidney | 60 years | 80084865 | 0.797 | 50 | E-MTAB-513 (ArrayExpress |
| | | | | | Archive, Illumina Human Body |
| | | | | | Map 2 project) |
| Liver | 37 years | 78751250 | 0.845 | 50 | E-MTAB-513 (ArrayExpress |
| | | | | | Archive, Illumina Human Body |
| | | | | | Map 2 project) |
| Lung | 65 years | 80276172 | 0.833 | 50 | E-MTAB-513 (ArrayExpress |
| | | | | | Archive, Illumina Human Body |
| | | | | | Map 2 project) |
| Lympho | 86 years | 81997309 | 0.798 | 50 | E-MTAB-513 (ArrayExpress |
| node | | | | | Archive, Illumina Human Body |
| | | | | | Map 2 project) |
| Muscle | 77 years | 82487888 | 0.844 | 50 | E-MTAB-513 (ArrayExpress |
| | | | | | Archive, Illumina Human Body |
| | | | | | Map 2 project) |
| Ovary | 47 years | 80974656 | 0.831 | 50 | E-MTAB-513 (ArrayExpress |
| | | | | | Archive, Illumina Human Body |
| | | | | | Map 2 project) |
| Prostate | 73 years | 82826989 | 0.846 | 50 | E-MTAB-513 (ArrayExpress |
| | | | | | Archive, Illumina Human Body |
| | | | | | Map 2 project) |
| Testis | 19 years | 81940259 | 0.848 | 50 | E-MTAB-513 (ArrayExpress |
| | | | | | Archive, Illumina Human Body |
| | | | | | Map 2 project) |
| Thyroid | 60 years | 81079772 | 0.852 | 50 | E-MTAB-513 (ArrayExpress |
| | | | | | Archive, Illumina Human Body |
| | | | | | Map 2 project) |

### RNA-seq Read Alignment, Gene Count Calculation and Normalization

Raw fastq files were downloaded with the fastq-dump function of the SRA-toolkit (v 2.8.0). This study mapped reads with STAR (v2.5.1a) using an index based on the soft masked primary assembly of reference genome GRCh38 and corresponding gene annotation gtf file (GRCh38.83). Both were obtained from the Ensembl FTP site. Read overhang was set to 99 bp for index generation. Default mapping parameters were retained with the following exceptions: “—outFilterType BySJout” to reduce the number of spurious junctions, “—alignSJoverhangMin 10” minimum read overhang for unannotated junctions, “—alignSJDBoverhangMin 1” minimum overhang for annotated junctions, “—outFilterMatchNminOverLread 0.95” to allow up to 5% mismatched bases (per pair) if no better alignment can be found, “—align IntronMin 20” to allow short introns, “—alignlntronMax 2000000” to set an upper limit on intron length, “—outMultimapperOrder Random” to randomize the choice of the primary alignment from the highest scoring alignments, “—outFilterIntronMotifs RemoveNoncanonicalUnannotated” to bias mapping towards known transcripts and “—chimSegmentMin 0” to suppress any chimeric mapping output. The mapped reads of all samples were then jointly processed with featureCounts as implemented in the package “Rsubread” (v1.16.1) in R (v3.1.2). Default settings were used with the following exceptions: “annot.ext=GTFfile, isGTFAnnotationFile=TRUE, GTF.featureType=‘exon’” to use the same gtf annotation file as in STAR index, “useMetaFeatures=TRUE, GTF.attrType=‘gene’” to summarize counts to the gene level, “allowMultiOverlap=TRUE” to allow counting in overlapping genes, “isPairedEnd” was set as appropriate for the respective samples, “strandSpecific=0” because not all libraries were strand-specific and finally “countMultiMappingReads=TRUE”. The resulting count table was normalized to account for sequencing depth and count distribution with the TMM method as implemented in edgeR (v3.8.6) using default settings.

### Bioinformatics Analysis

RNA-seq gene expression analysis was carried out using normalized counts for each gene in each tissue type. Where technical replicates are available for samples described in other studies, these reads were aligned and gene counts determined separately, then average gene counts were calculated. Furthermore, unless otherwise stated, gene counts for cPP and PPd15 cells are the mean of three independent samples harvested from cells derived from H9 and HES3 hESC, and AK6-13 hiPSC. For global comparisons of gene expression profiles, we compared 60,675 ENSEMBL genes or (where stated) 19,875 ENSEMBL protein-coding genes expressed at >5 normalized counts in at least one sample. All of the following analysis was carried out in R, using base packages unless stated otherwise.

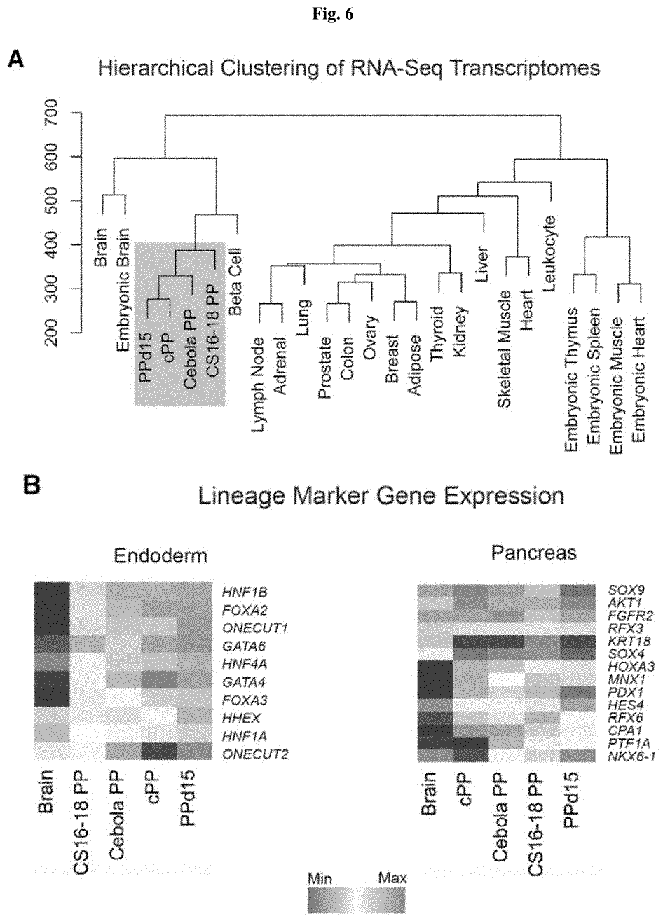

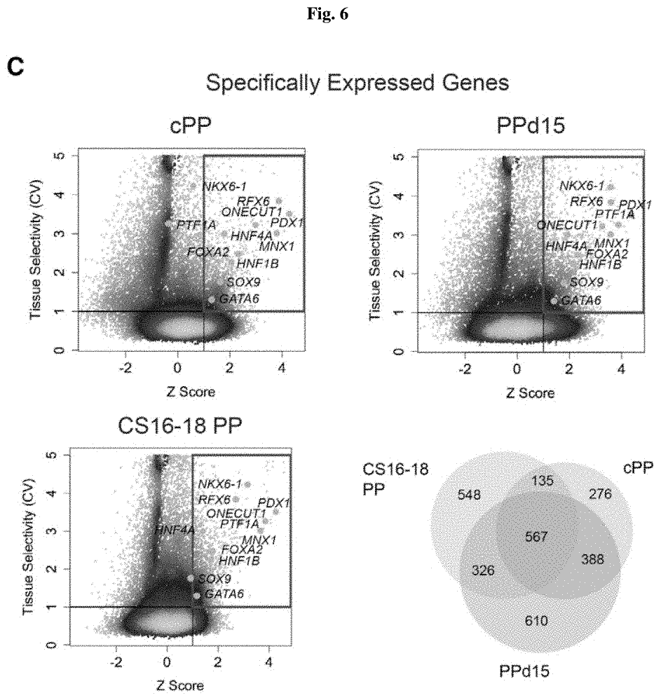

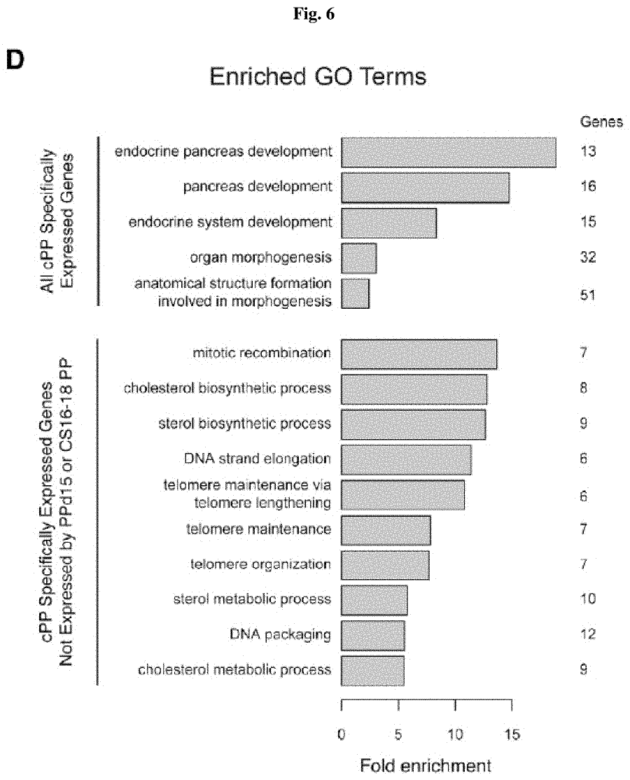

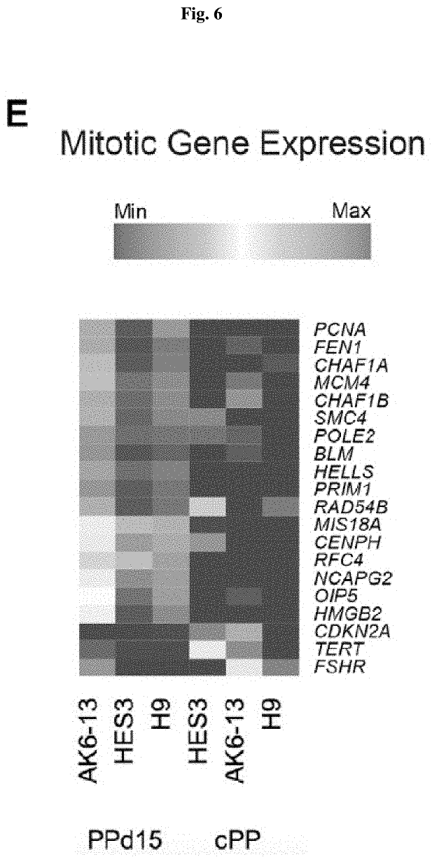

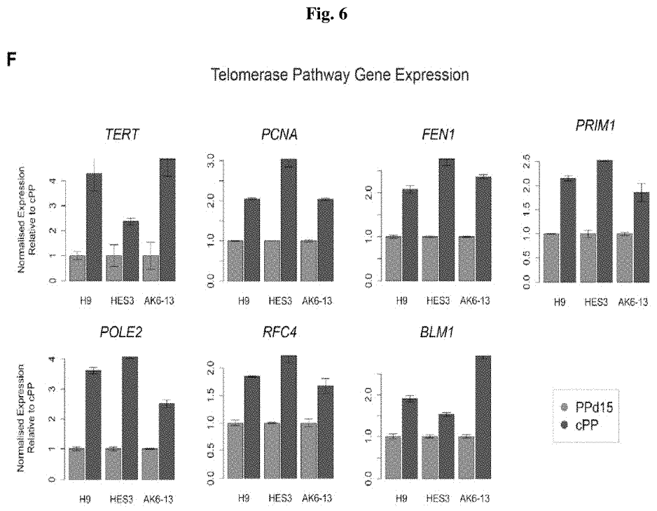

### Hierarchical Clustering of RNA-Seq Transcriptomes (FIG. 6A)

Euclidian distances between pairs of log2-transformed global gene counts were calculated using the R function dist( )and the distances plotted as a Dendrogram using the hclust( )function.

### Heatmaps (FIG. 6B, 6E and 6F)

Heatmaps were plotted using the function heatmap.2( ).

### Specifically Expressed Genes (FIG. 6C)

Specifically expressed genes are defined as those with CV >1 (Coefficient of Variance) and Z-score >1. CV is defined as the mean divided by the standard deviation across all samples, in this case the aforementioned 23 published tissue datasets plus the cPP and PPd15 gene counts described here. Zscore is defined as the difference between expression in the sample of interest and the mean for all samples, divided by the standard deviation across all samples. When calculating the Z-score for pancreatic samples other pancreatic samples are excluded.

### Gene Ontogeny Analysis (FIG. 6D)

The web-based gene set analysis tool kit at http://www.webgestalt.org/ was used to analyze Gene Ontogeny (GO) terms associated with genes specifically expressed by cPP cells. Protein-coding genes were ordered according to the product of the coefficient of variance and Z-score for cPP cells (see above) and the top 250 genes selected for enrichment analysis. The Over Representations Analysis (ORA) tool was used to calculate fold-enrichment for biological process GO terms across these 250 genes, using all protein coding genes as the reference set, and the corresponding p-value adjusted by the Benjamini-Hochberg multiple test adjustment. GO terms were ordered according to fold enrichment and those associated with <5 genes and/or an adjusted p-value >0.01 were eliminated from the enriched set.

### Multilineage Differentiation

Monolayer differentiation cultures were established as described herein. Basal differentiation medium consists of advanced DMEM/F12 (Thermo Fisher Scientific, 21634010), 2.5 g/30 mL BSA (Sigma, A9418), 2 mM L-glutamine (Thermo Fisher Scientific, 25030), 100 U/mL penicillin/streptomycin (Thermo Fisher Scientific, 15140122), and 1× B27 supplement (Thermo Fisher Scientific, 17504-044). Supplements were added as follows: days 1-3 (3 μM RA [Sigma, R2625], 1 μM DAPT [Sigma, D5942], 100 μM BNZ [Sigma, B4560]) and days 4, 7, and 10 (3 μM RA, 167 ng/mL KAAD-cyclopamine [Calbiochem, 239807]).

In vitro Differentiation

### Establishing Differentiation Cultures

Initially, cPP cells were cultured to confluency to eliminate feeder cells then treated with gentle cell dissociation reagent to generate single cells. Single cells were resuspended in cPP culture media +10 μM Y27632 and seeded according to differentiation platform. To establish 3D sphere cultures, 2×10⁶ cells were seeded into each well of an ultra-low adhesion 6 well plate (Corning, 3471) in 2 mL media and placed on a nutator overnight. Compact spheres typically form after 24 hours. To establish 3D matrigel cultures, AggreWell 400 plates (Stemcell Technologies, 27840) were used to generate spheres of ˜200 cells according to the manufacturer's instructions. After 24 hours ˜1200 spheres were resuspended in 500 μL 1:5-diluted hESC-qualified matrigel (Corning, 354277) and deposited into each well of a 24 well plate. Plates were incubated at 37° C. for 60 min to allow matrigel to solidify before addition of media. To establish 2D monolayer cultures, cells were seeded at 6.65×10⁵cells/cm² on tissue culture plastic coated with matrigel diluted 1:50.

### NKX6-1 Induction Tests

Differentiation cultures were treated with the following signaling regimes, based upon several recently published protocols, with minor alterations (Pagliuca et al., 2014; Rezania et al., 2014; Russ et al., 2015; Zhang et al., 2009). Differentiation media 1 consists of MCDB 131 media (Thermo Fisher Scientific, 10372-01), 2.5 g/L sodium bicarbonate (Lonza, 17-613E), 2 mM L-glutamine, 100 U/mL penicillin/streptomycin, 10 mM glucose (VWR International, 101174Y), and 2% bovine serum albumin (Sigma, A9418). Differentiation media 2 consists of DMEM high glucose, 2 mM L-glutamine, and 100 U/mL penicillin/streptomycin. Media based on PP2 induction media described by Pagliuca et al. consists of differentiation media 1 supplemented with 50 ng/mL FGF7 (R&D Systems, 251-KG), 0.25 mM ascorbic acid (Sigma, A4544), 100 nM RA, 0.25 μM SANT-1 (Sigma, S4572), and 0.5% ITS-X (Thermo Fisher Scientific, 51500056). Media was completely replenished daily for 5 days. Media based on stage 4 media described by Rezania et al. was additionally supplemented with 300 nM lndolactam-V (Stemcell Technologies, 72312) and 200 nM LDN-193189 (Stemcell Technologies, 72142), and was completely replenished daily for 5 days. Media based on day 13-20 media described by Zhang et al. consists of differentiation media 2 supplemented with 10 ng/mL bFGF, 10 mM nicotinamide (Sigma, 24,020-6), 50 ng/mL exendin-4 (Sigma, E7144), 10 ng/mL BMP4 (R&D Systems, 314-BP), and 1% ITS-X. Media was completely replenished daily for 5 days. Media based on day 7-9 media described by Russ et al. consists of differentiation media 2 supplemented with 1×B27 supplement, 50 ng/mL EGF, 1 μM RA (first 24 hours), and 50 ng/mL FGF7 (second 24 hours). Media was completely replenished daily for 2 days.

### β Cell Differentiation

Differentiation sphere cultures were established as described in herein. Basal differentiation medium consists of DMEM high glucose, 2 mM L-glutamine, and 100 U/mL penicillin/streptomycin. Supplements were added as follows: days 1-4 (1×B27 supplement, 50 ng/mL EGF, 1 μM RA [days 1-2 only], 50 ng/mL FGF7 [days 3-4 only]); days 5-10 (1× B27 supplement, 500 nM LDN-193189 [STEMCELL Technologies, 72142], 30 nM TPB [EMD Millipore, 565740], 1 μM RepSox [STEMCELL Technologies, 72392], 25 ng/mL FGF7); and days 11-17 (DMEM low glucose [Thermo Fisher Scientific, 12320-032], 2 mM L-glutamine, 1× MEM non-essential amino acids [Thermo Fisher Scientific, 11140-050]).

### Transplantation Assays

cPP cells were grown to confluency to displace and eliminate feeder cells, then treated with gentle cell dissociation reagent to generate single cells. Approximately 3×10⁶ to 5×10⁶ cells were resuspended in 50 μL of undiluted Matrigel and injected under the kidney capsule of 8- to 12-week-old immunocompromised (NOD/SCID) mice. After 23-27 weeks, transplanted mice were euthanized and their kidneys cryopreserved prior to sectioning and immunostaining. The study protocol was approved by the National University of Singapore Institutional Review Board (NUS IRB 12-181) and Biomedical Research Council IACUC committee (151040).

### Quantitative RT-PCR

RNA was isolated from samples using an RNeasy mini kit (Qiagen, cat # 74104) and reverse transcribed to generate cDNA using a high-capacity reverse transcription kit and random hexamer primers (Applied Biosystems, 4368814,1 μg RNA per 20 μL reaction). Quantitative RT-PCR was carried out using SYBR Select Mastermix (Applied Biosystems, 4472908). Data were analyzed using the ΔΔCT method, and normalized to expression of the housekeeping gene TBP in each sample. The primers used for qRT-PCR are shown in Table 2.

TABLE 2
Primers use for qRT-PCR.

| Name | Sequence | SEQ ID |
|---|---|---|
| AMY2B-Forward | ATGCCTTCTGACAGAGCACT | SEQ ID NO.: 1 |
| AMY2B-Reverse | ACAGCCTAGCATCCCAGAAG | SEQ ID NO.: 2 |
| BLM-Forward | CAGACTCCGAAGGAAGTTGTATG | SEQ ID NO.: 3 |
| BLM-Reverse | TTTGGGGTGGTGTAACAAATGAT | SEQ ID NO.: 4 |
| CAII-Forward | GCCAAGTATGACCCTTCCCT | SEQ ID NO.: 5 |
| CAII-Reverse | CCACGTTGAAAGCATGACCA | SEQ ID NO.: 6 |
| CPA1-Forward | CTGACCATCATGGAGCACAC | SEQ ID NO.: 7 |
| CPA1-Reverse | GCCAGAGAGGAGGACAAGAA | SEQ ID NO.: 8 |
| FEN1-Forward | ATGACATCAAGAGCTACTTTGGC | SEQ ID NO.: 9 |
| FEN1-Reverse | GGCGAACAGCAATCAGGAACT | SEQ ID NO.: 10 |
| FOXA2_Forward | GGGAGCGGTGAAGATGGA | SEQ ID NO.: 11 |
| FOXA2_Reverse | TCATGTTGCTCACGGAGGAGTA | SEQ ID NO.: 12 |
| GATA4_Forward | TCCCTCTTCCCTCCTCAAAT | SEQ ID NO.: 13 |
| GATA4_Reverse | TCAGCGTGTAAAGGCATCTG | SEQ ID NO.: 14 |
| GATA6_Forward | CAGTTCCTACGCTTCGCATC | SEQ ID NO.: 15 |
| GATA6_Reverse | TTGGTCGAGGTCAGTGAACA | SEQ ID NO.: 16 |
| GCG_Forward | AAGCATTTACTTTGTGGCTGGATT | SEQ ID NO.: 17 |
| GCG_Reverse | TGATCTGGATTTCTCCTCTGTGTCT | SEQ ID NO.: 18 |
| HNForwardB_Forward | TCACAGATACCAGCAGCATCAGT | SEQ ID NO.: 19 |
| HNForwardB_Reverse | GGGCATCACCAGGCTTGTA | SEQ ID NO.: 20 |
| HNF4A_Forward | CATGGCCAAGATTGACAACCT | SEQ ID NO.: 21 |
| HNF4A_Reverse | TTCCCATATGTTCCTGCATCAG | SEQ ID NO.: 22 |
| INS_Forward | CAGGAGGCGCATCCACA | SEQ ID NO.: 23 |
| INS_Reverse | AAGAGGCCATCAAGCAGATCA | SEQ ID NO.: 24 |
| ISL1-Forward | AAACAGGAGCTCCAGCAAAA | SEQ ID NO.: 25 |
| ISL1-Reverse | AGCTACAGGACAGGCCAAGA | SEQ ID NO.: 26 |
| KRT19-Forward | AACGGCGAGCTAGAGGTGA | SEQ ID NO.: 27 |
| KRT19-Reverse | GGATGGTCGTGTAGTAGTGGC | SEQ ID NO.: 28 |
| NGN3_Forward | GCTCATCGCTCTCTATTCTTTTGC | SEQ ID NO.: 29 |
| NGN3_Reverse | GGTTGAGGCGTCATCCTTTCT | SEQ ID NO.: 30 |
| NKX2-2_Forward | GGGACTTGGAGCTTGAGTCCT | SEQ ID NO.: 31 |
| NKX2-2_Reverse | GGCCTTCAGTACTCCCTGCA | SEQ ID NO.: 32 |
| NKX6-1_Forward | CACACGAGACCCACTTTTTC | SEQ ID NO.: 33 |
| NKX6-1_Reverse | CCGCCAAGTATTTTGTTTGT | SEQ ID NO.: 34 |
| ONECUT1_Forward | GTGTTGCCTCTATCCTTCCCAT | SEQ ID NO.: 35 |
| ONECUT1_Reverse | CGCTCCGCTTAGCAGCAT | SEQ ID NO.: 36 |
| PCNA-Forward | CCTGCTGGGATATTAGCTCCA | SEQ ID NO.: 37 |
| PCNA-Reverse | CAGCGGTAGGTGTCGAAGC | SEQ ID NO.: 38 |
| PDX1_Forward | AAGTCTACCAAAGCTCACGCG | SEQ ID NO.: 39 |
| PDX1_Reverse | GTAGGCGCCGCCTGC | SEQ ID NO.: 40 |
| POLE2-Forward | TGAGAAGCAACCCTTGTCATC | SEQ ID NO.: 41 |
| POLE2-Reverse | TCATCAACAGACTGACTGCATTC | SEQ ID NO.: 42 |
| PRIM1-Forward | ATGGAGACGTTTGACCCCAC | SEQ ID NO.: 43 |
| PRIMI-Reverse | CGTAGTTGAGCCAGCGATAGT | SEQ ID NO.: 44 |
| RFC4-Forward | CCGCTGACCAAGGATCGAG | SEQ ID NO.: 45 |
| RFC4-Reverse | AGGGAACGGGTTTGGCTTTC | SEQ ID NO.: 46 |
| RFX6_Forward | AGCGGATCAATACCTGTCTCAGAA | SEQ ID NO.: 47 |
| RFX6_Reverse | GCATAAAGAATGCACCGTGGTAAG | SEQ ID NO.: 48 |
| SOX9_Forward | GAACGCACATCAAGACGGAG | SEQ ID NO.: 49 |
| SOX9_Reverse | AGTTCTGGTGGTCGGTGTAG | SEQ ID NO.: 50 |
| SST_Forward | CCCCAGACTCCGTCAGTTTC | SEQ ID NO.: 51 |
| SST_Reverse | TCCGTCTGGTTGGGTTCAG | SEQ ID NO.: 52 |
| TBP_Forward | TATAATCCCAAGCGGTTTGC | SEQ ID NO.: 53 |
| TBP_Reverse | GCACACCATTTTCCCAGAAC | SEQ ID NO.: 54 |
| TERT-Forward | AAATGCGGCCCCTGTTTCT | SEQ ID NO.: 55 |
| TERT-Reverse | CAGTGCGTCTTGAGGAGCA | SEQ ID NO.: 56 |
| TRYP3-Forward | CATCAATGCGGCCAAGATCA | SEQ ID NO.: 57 |
| TRYP3-Reverse | GGAATTGATGACGGCAGGTG | SEQ ID NO.: 58 |

### Immunofluorescence Staining

The following primary antibodies were used for immunofluorescence staining: mouse monoclonal anti-PDX1 (R&D Systems, MAB2419, 1:50), rabbit anti-SOX9 (Sigma, HPA001758, 1:2000), rabbit anti-HNF6 (ONECUT1) (Santa Cruz, S.C.13050, 1:100), goat anti-FOXA2 (R&D Systems, AF2400, 1:200), rabbit anti-GATA6 (Cell Signaling Technologies, 5851, 1:1600), sheep anti-NGN3 (R&D Systems, AF3444, 1:200), mouse anti-NKX6-1 (developmental studies hybridoma bank, F55A12, 1:80), mouse monoclonal anti-KX2-2 (BD biosciences, 564731, 1:400), mouse monoclonal anti-pro-Insulin cpeptide (Millipore, 05-1109, 1:100), rabbit monoclonal anti-glucagon (Cell Signaling Technologies, 8233, 1:400), rat monoclonal anti-KRT19 (developmental studies hybridoma bank, TROMA-III-s, 1:10), sheep anti-trypsin (pan-specific) (R&D Systems, AF3586, 1:13). Primary antibodies were recognized by Alexa-fluorophore conjugated secondary antibodies raised in Donkey (Thermo Fisher Scientific, 1:500). Images were acquired using an Olympus FV1000 inverted confocal microscope.

### Immunofluorescence Staining Transplanted Kidneys

Mouse kidneys were dissected, cleaned, longitudinally sectioned, embedded in Jung freezing medium (Leica, 020108926), and cryopreserved in liquid nitrogen. Sections (6 μm) were mounted on APEScoated glass slides, dried and fixed in 4% paraformaldehyde for 10 min at room temperature. After washing 3× with PBS for 15 min, samples were permeabilised with PBS containing 0.3% Triton X-100 for 10 min, then blocked for 1 hour each in Rodent block M (Biocare medical, RBM961 H) and blocking buffer (PBS+20% normal donkey serum+1% BSA+0.3% Triton X-100). After washing 3× with wash buffer (PBS+0.1% Tween-20+0.1% BSA) for 15 min, samples were incubated overnight at 4oC with primary antibodies diluted in blocking buffer. After washing 3× with wash buffer for 15 min, samples were incubated at room temperature for 1 hour with secondary antibodies diluted 1:500 in blocking buffer. All subsequent steps were carried out in the dark. After washing 1× with wash buffer, samples were incubated at room temperature for 20 min with 2 μg/mL Hoechst-33342 (Thermo Fisher Scientific, 62249) diluted in PBS. Finally, after washing 3× with wash buffer for 15 min, samples were covered with Vectashield hard set mounting medium (Vector Laboratories, H-1400), covered with a coverslip and sealed.

### Immunofluorescence Staining Cultured Cells

Adherent cells were washed 2× with PBS then fixed in 4% paraformaldehyde for 20 min at room temperature. After washing 3× with wash buffer (PBS +0.1% BSA), samples were incubated with blocking buffer (PBS+20% normal donkey serum+0.1% BSA+0.3% Triton X-100) for 1 hour at room temperature. Samples were then incubated overnight at 4oC with primary antibodies diluted in blocking buffer. After washing 3× with wash buffer for 15 min, samples were incubated at room temperature for 1 hour with secondary antibodies diluted 1:500 in blocking buffer. All subsequent steps were carried out in the dark. After washing 3× with wash buffer for 15 min, samples were incubated at room temperature for 15 min with 2 μg/mL Hoechst-33342 (Thermo Fisher Scientific, 62249) diluted in PBS. Finally, samples were washed 2× with PBS for 15 min and imaged.

### Immunofluorescence Staining Differentiation Spheres

Differentiation spheres were washed 1× with PBS+2% serum then fixed in 4% paraformaldehyde for 30 min at room temperature. After washing 1× for 15 min with wash buffer (PBS+0.1% BSA+0.1% Tween-20), samples were blocked for 6 hours in blocking buffer (PBS+20% normal donkey serum+1% BSA+0.3% Triton X-100). Samples were then incubated overnight at 4oC with primary antibodies diluted in blocking buffer. After washing 2× with wash buffer for 15 min, samples were incubated at 4° C. for 6 hours with secondary antibodies diluted 1:500 in blocking buffer. All subsequent steps were carried out in the dark. After washing 1× with wash buffer for 15 min, samples were incubated at room temperature for 1 hour with 2 μg/mL Hoechst-33342 (Thermo Fisher Scientific, 62249) diluted in PBS. Finally, spheres were washed 2× with PBS for 30 min, resuspended in Vectashield hard set mounting medium (Vector Laboratories, H-1400), mounted on glass slides, covered with a coverslip and sealed. All washing and incubation steps are carried out in 1.5mL Eppendorf tubes.

### Flow Cytometry

Single cells were generated using accutase (Thermo Fisher Scientific, 14190), washed 1× with PBS+1% serum, then fixed in 4% paraformaldehyde for 10 min at room temperature. Cells were washed 1× with wash/permeabilization buffer (BD, 554723), then up to 10⁶ cells were incubated with primary or isotype control antibody diluted in 250 μL wash/permeabilization buffer for the required length of time (see below for antibody dilutions and incubation times). For unconjugated antibodies, cells were washed 1× with wash/permeabilization buffer then incubated for 15 min with secondary antibody diluted in wash/permeabilization buffer. If staining for a second antigen, cells were washed 1× with wash/permeabilization buffer then subject to the aforementioned incubation step(s). After washing 1× with wash/permeabilization buffer, cells were resuspend cells in PBS+1% serum and analyzed using a BD FACSCalibur flow cytometer. All steps were carried out at room temperature and cells were pelleted by centrifugation at 6000 rpm for 5 min in a microcentrifuge.

The following antibodies were used: mouse monoclonal anti-PDX1 PE-conjugate (BD biosciences, 562161, 1:50, 45 min), mouse IgG1 PE-conjugate (BD biosciences, 556650, 1:50, 45 min), mouse monoclonal anti-NKX6.1 (developmental studies hybridoma bank, F55A12, 1:25, 45 min), goat antimouse IgG APC-conjugate (BD biosciences, 550828, 1:400, 15 min), mouse monoclonal anti-October3/4 Alexa Fluor 488-conjugate (BD biosciences, 560253, 1:5, 60 min), mouse monoclonal anti-pro-Insulin c-peptide (Millipore, 05-1109, 1:100, 60 min), anti-mouse IgG Alexa Fluor 488-conjugate (Thermo Fisher Scientific, A21202, 1:300, 30 min). All flow cytometry experiments were gated using cells stained only with fluorophore-conjugated isotype control (in the case of directly conjugated primary antibodies) or fluorophore-conjugated secondary antibodies.

### Microbioreactor Array (MBA) Screening of cPP Maintenance and Proliferation

Microbioreactor arrays were used to screen the effects of combinations of exogenous signaling molecules on cPP cells. MBAs provide combinatorial mixing of input factors, combined with continuous flow of culture media over culture chambers. MBAs were autoclaved and filled with sterile PBS, then coated (2-4 h, room temperature) with a single 1 mL injection of hESC-qualified matrigel at the manufacturer's recommended concentration. cPP cells in suspension in complete medium at 5×106/mL were then seeded in the MBA, giving a surface density of 50×10⁶cells/cm². Cells were allowed to attach for a total of 20 h, with a media exchange performed every 6 h. Subsequently, factor provision was commenced with an initial filling step of 300pL, followed by constant perfusion of factors at 36 μL/h, for a total culture time of 3 days. At the endpoint, cells were rinsed with PBS, fixed with 2% PFA/PBS solution for 30 min, then rinsed with PBS and blocked/permeabilised with PBS+20% normal donkey serum+0.1% BSA+0.3% Triton X-100 for 30 min. Then, cells were labeled with primary antibodies against PDX1 (R&D Systems, MAB2419, 1:25), and SOX9 (Sigma, HPA001758, 1:1000) diluted in blocking buffer, overnight at 4° C. Cells were then washed with 0.1% BSA/PBS and labeled with Alexa-fluorophore conjugated secondary antibodies (Thermo Fisher Scientific, 1:500 dilution) and Hoechst 33342 (2 pg/mL) for 1 hour. Finally, cells were rinsed with PBS, and the MBA inlets and outlets plugged closed. The MBA was then mounted in a microplate adapter and imaged. Nuclear segmentation and quantification of nuclear intensities of PDX1 and SOX9 then proceeded similarly as previously described.

### Accession Numbers

Primary RNA-seq datasets generated in this study are available at ArrayExpress under accession number ArrayExpress: E-MTAB-5731.

### RESULTS

Maintenance and Expansion of cPP Cells Derived from hESCs and hiPSCs

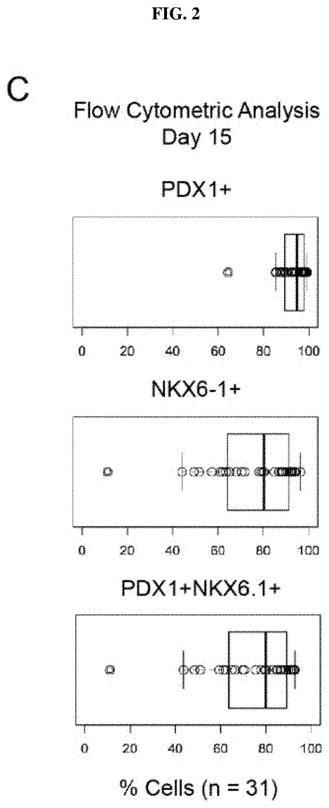

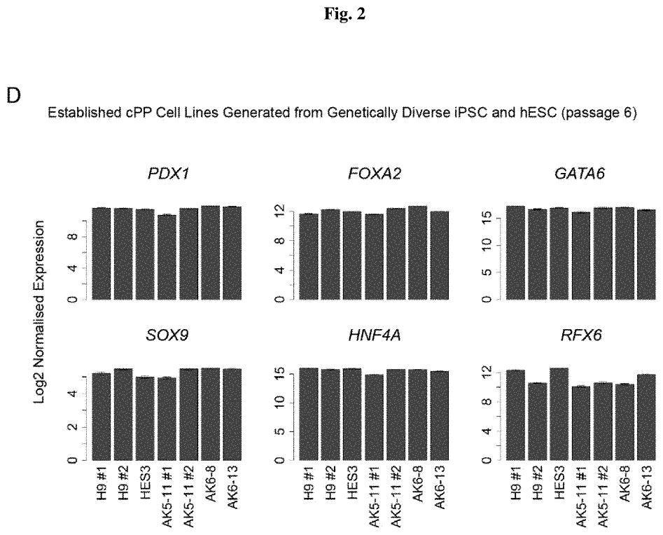

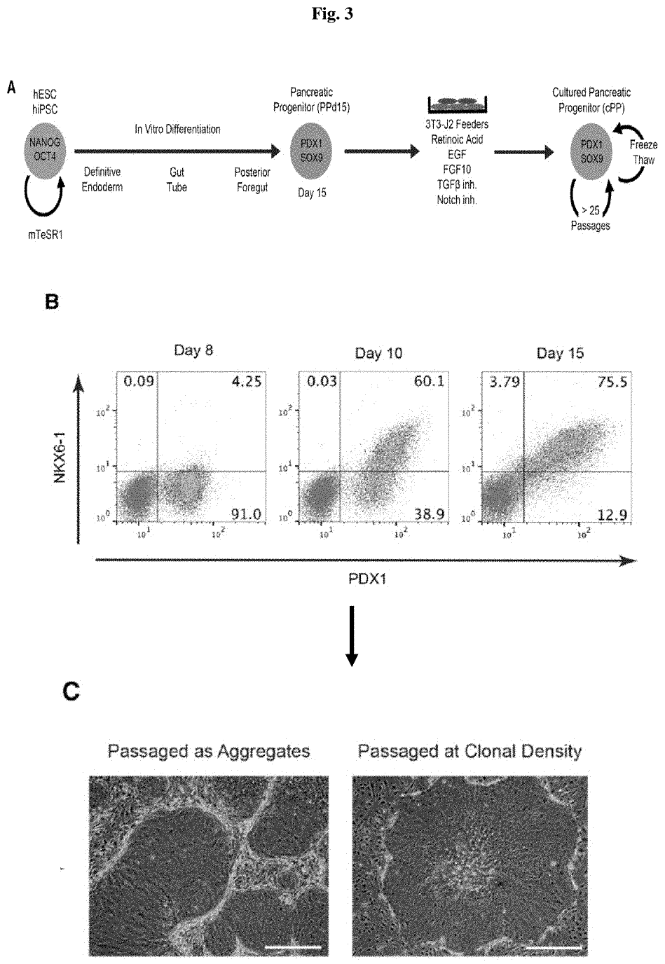

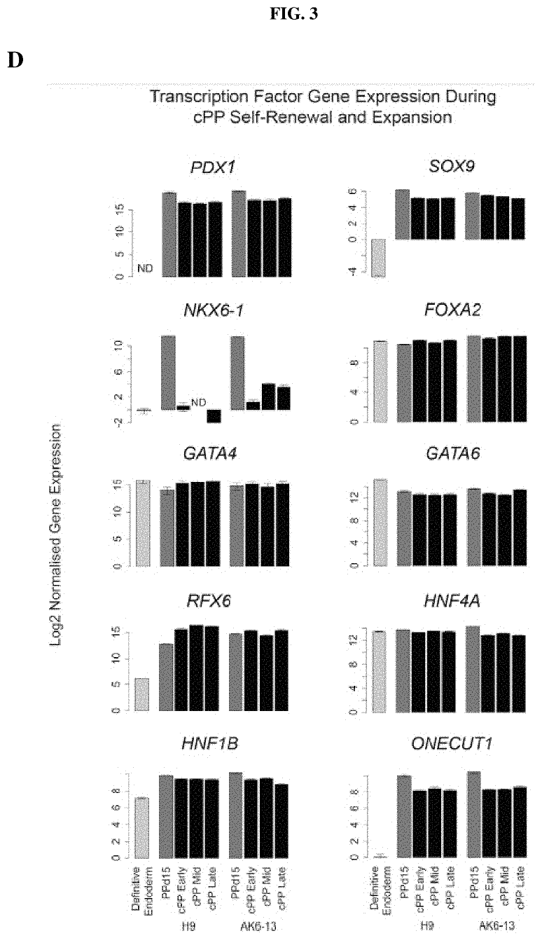

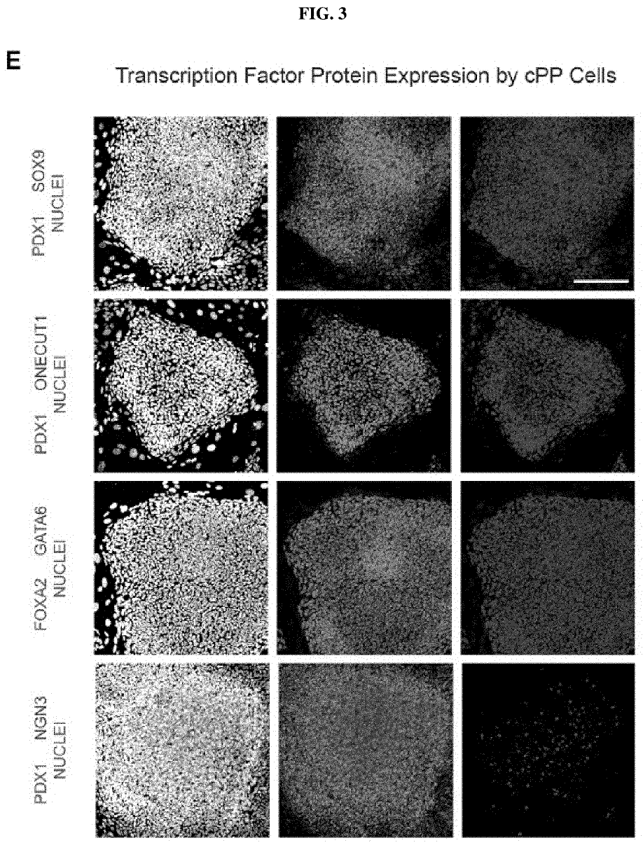

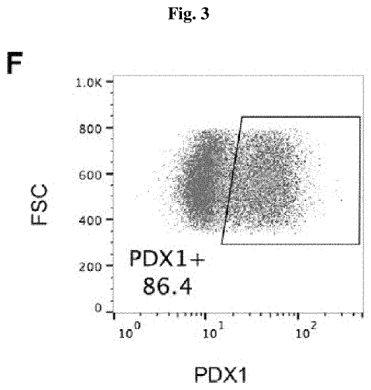

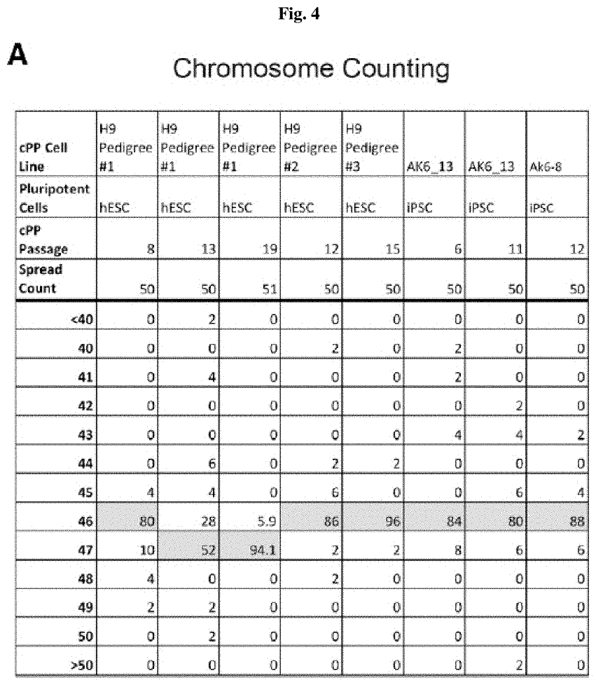

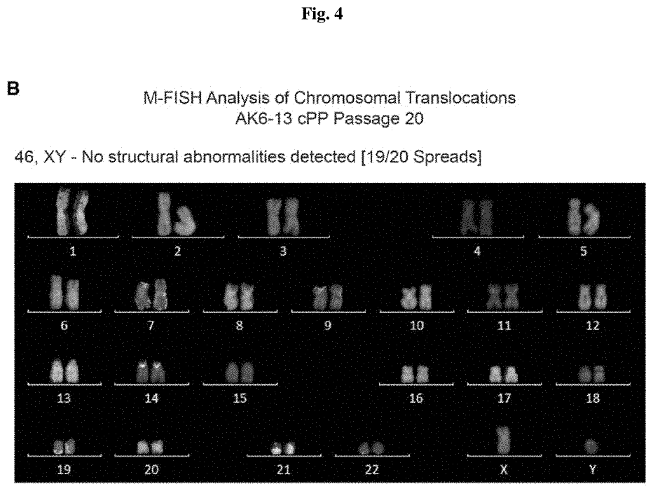

Directed differentiation guided by growth factors and small molecules facilitates the generation of diverse cell types from pluripotent stem cells. Pancreatic progenitors were produced from hESCs and hiPSCs (FIG. 1) using reagents based on the early stages of a protocol designed to generate mature β cells (FIG. 2A; Rezania et al., 2014). This differentiation strategy induced the sequential expression of PDX1 followed by NKX6-1 and yielded a median of 80% PDX1+NKX6-1+ cells after 15 days (PPd15 cells; FIG. 3B and 2C). However, as is often observed during directed differentiation from pluripotent cells, the kinetics of PDX1 and NKX6-1 expression varied between cell lines (FIG. 2B). Therefore, this study sought to capture, synchronize, and expand PPd15 cells in culture.

The 3T3-J2 mouse embryonic fibroblast cell line has been used to culture progenitor cells derived from a variety of human tissues, including endoderm-derived intestinal stem cells. This study therefore determined whether pancreatic progenitor cells could be similarly expanded, if provided with appropriate stimuli. A series of signaling agonists and inhibitors previously shown to regulate pancreatic development were tested, including EGFL7, BMP4, nicotinamide, LIF, WNT3A, R-Spondin-1, Forskolin (cAMP agonist), GSK3b inhibition (CHIR99021), and inhibitors of BMP (LDN-193189) and SHH (KAAD-cyclopamine) signaling. Ultimately, a combination of EGF, retinoic acid, and inhibitors of transforming growth factor β (TGF-β, SB431542) and Notch signaling (DAPT) was found to support long-term self-renewal of pancreatic progenitors (FIG. 3A). To establish stable cPP cell lines, PPd15 cells were replated on a layer of 3T3-J2 feeder cells in the presence of these factors. Thereafter, cPP cells were routinely passaged once weekly as aggregates at an average split ratio of 1:6, although they were also capable of forming colonies at clonal density (FIG. 3C). This suggests a doubling time of ˜65 hr in culture, similar to the 61 hr routinely observed for hESCs when cultured on a layer of mouse embryonic fibroblasts.

This study was able to generate self-renewing cPP cell lines from four different genetic backgrounds using two hESC (H9 and HES3) and three hiPSC cell lines (AK5-11, AK6-8, and AK6-13 derived in house); these diverse cPP cells expressed comparable levels of genes encoding key pancreatic transcription factors, including PDX1 and SOX9 (FIG. 2D). Two cPP cell lines selected for further analysis (H9#1 and AK6-13) have been maintained in culture for >20 passages to date enabling >1018-fold expansion over 20 weeks. Crucially, cPP cells can be frozen and thawed with no apparent loss of proliferation or viability, suggesting cPP cells could replace pluripotent cells as a starting point for further differentiation to mature pancreatic cell types such as insulin-secreting β cells.

To determine whether cPP cultures consist of a stable and homogeneous population of cells, the expression of key pancreatic transcription factors was measured at the mRNA and protein levels. Gene expression of numerous markers of pancreatic bud cells, including PDX1 and SOX9, remained constant over extended periods in culture, indicating

that the culture conditions maintain a stable population of pancreatic progenitors (FIG. 3D).

To determine whether cPP cultures represent a homogeneous population, immunostaining was carried out for a selection of pancreatic markers and found these to be expressed near ubiquitously at the protein level (FIG. 3E). Furthermore, flow cytometric analysis showed that approximately 85% of cPP cells were PDX1+ (FIG. 3F).

However, NKX6-1 expression was rapidly downregulated in culture, and NKX6-1 protein was not detected by immunostaining. Furthermore, we were able to establish cPP cell lines from day 7, 10, and 15 differentiation cultures (data not shown), the earliest time point being prior to expression of NKX6-1 and suggesting that cPP culture conditions stabilize pancreatic progenitors in a developmental state that precedes NKX6-1 activation. Very few cells were NGN3+, which marks early endocrine progenitors, indicating that differentiation was blocked at the progenitor stage under our culture conditions. Finally, chromosome counting showed that five out of six cPP cells carried 46 chromosomes without signs of structural changes, such as presence of fragments or dicentric chromosomes (FIG. 4A). Multiplex fluorescence in situ hydridization (M-FISH) analysis on the AK6-13 line at passage 20 confirmed the absence of karyotypic abnormalities (FIG. 4B). Collectively, these data demonstrate that the cPP culture conditions capture pancreatic progenitors as a near homogeneous population that is maintained stably over extended periods of time and is capable of extensive expansion.

### Transcriptome Analysis Demonstrates cPP Cells Are Closely Related to Their In Vivo Counterparts

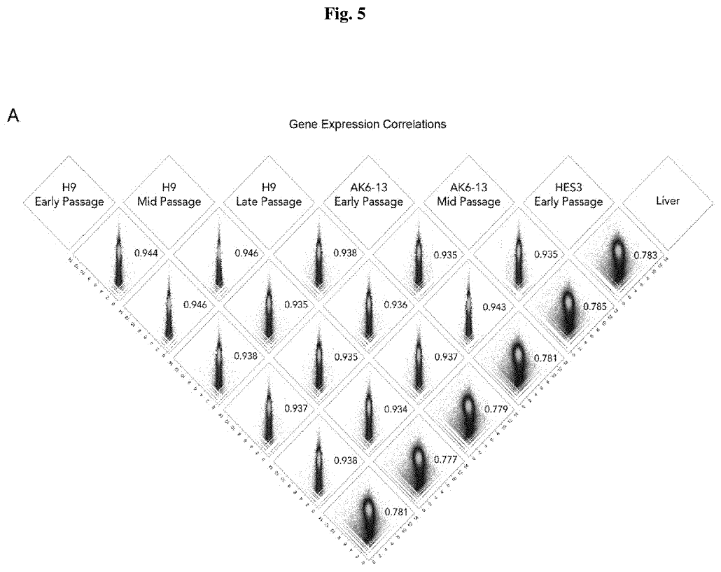

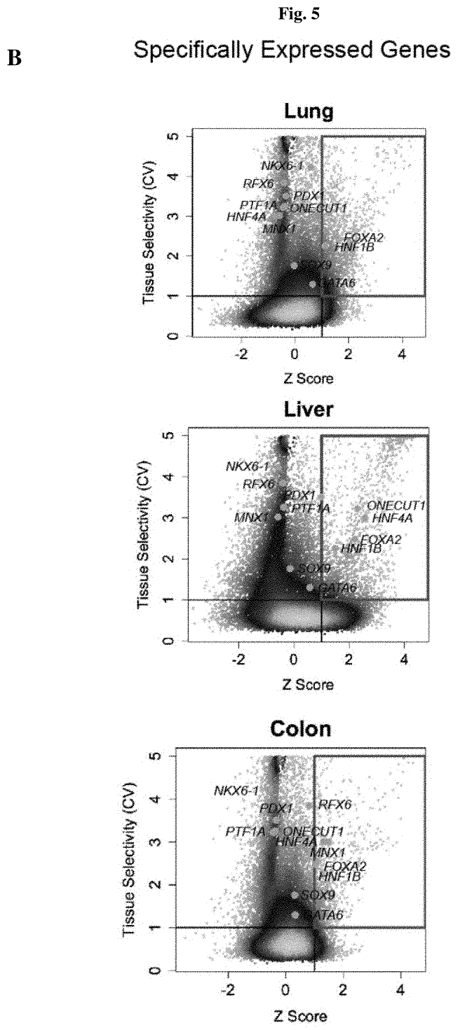

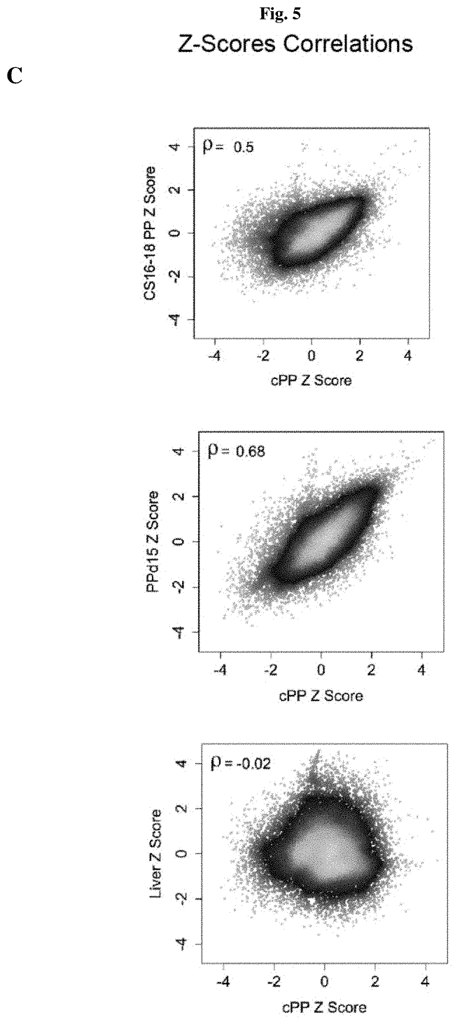

Next, this study determined the transcriptome-wide gene counts by RNA-seq for cPP lines from three different genetic backgrounds and the PPd15 differentiation cultures from which they were established. Samples for RNA-seq were also taken from cPP cells at early, mid, and late passages. Gene expression levels correlated strongly between different cPP samples, indicating that neither genetic background nor time in culture significantly affect the cPP transcriptome (FIG. 5A). However, to completely eliminate donor-specific effects on gene expression, the following analysis used mean gene counts for cPP (early passage) and PPd15 cells derived from H9 and HES3 hESCs and AK6-13 hiPSCs.

To determine how similar cPP cells are to their in vitro and in vivo counterparts, the cPP transcriptome was compared with the published transcriptomes of pancreatic progenitors differentiated in vitro (Cebola PP) and from CS16-18 human embryos (CS16-18 PP), and a diverse collection of adult and embryonic tissues. Relative to non-pancreatic tissues, cPP cells exhibited similar patterns of gene expression to both PPd15 and Cebola PP cells (FIG. 6A). Furthermore, cPP, PPd15, and Cebola PP cells closely resembled in vivo pancreatic progenitors at CS16-18, and all four cell populations expressed similar levels of genes associated with endodermal and pancreatic development (FIG. 6B). However, as expected, cPP cells do not express the late-stage pancreatic progenitor markers NKX6-1, PTF1A, and CPA1. When taken together, these data demonstrate that the culture conditions described here maintain cPP cells in a developmental state closely related to both the embryonic human pancreas and pancreatic progenitors generated by directed differentiation.

To further characterize the transcriptional identity of cPP cells, this study sought to identify genes that distinguish them from other lineages. Specifically expressed genes were defined as those that are variably expressed across the aforementioned panel of 25 tissues (coefficient of variance >1) and whose expression is upregulated in cPP cells (Z score >1). In total 1,366 genes were identified, including numerous well-characterized markers of pancreatic progenitor cells, such as PDX1, SOX9, MNX1, and RFX6 (FIG. 6C). To confirm the validity of this method, this study demonstrated that these genes are not expressed by other endodermal derivatives, including liver, colon, and lung (FIG. 5B). Encouragingly, around 80% of genes specifically expressed by cPP cells were shared with CS16-18 pancreatic progenitors and/or PPd15 cells. Furthermore, gene Z scores were highly correlated between these three pancreatic cell types but not with liver (FIG. 5C), further demonstrating the transcriptional similarities between cPP cells and other pancreatic progenitors.

To determine the functional roles of cPP-specific genes, this study analyzed associated Gene Ontology (GO) terms. The most enriched terms were those associated with endocrine pancreas development (FIG. 3D, above). In order to determine how the culture conditions affect the behavior of cPP cells, this study analyzed GO terms associated with genes expressed by cPP cells but not PPd15 or CS16-18 pancreatic progenitor cells (FIG. 6D, below). Interestingly, the most enriched terms were those associated with aspects of cell division and telomere maintenance. Indeed, genes associated with these enriched terms, such as those encoding telomerase reverse transcriptase (TERT) and proliferating cell nuclear antigen (PCNA), were consistently upregulated in cPP cells from different genetic backgrounds, compared with the PPd15 populations from which they were derived (FIG. 6E and 6F). The inventors concluded that the feeder-based culture system maintains pancreatic progenitors as a stable population while upregulating genes required for long-term self-renewal.

A Feeder Layer of 3T3-J2 Cells Prevents cPP Differentiation while Exogenous Signals Promote Proliferation

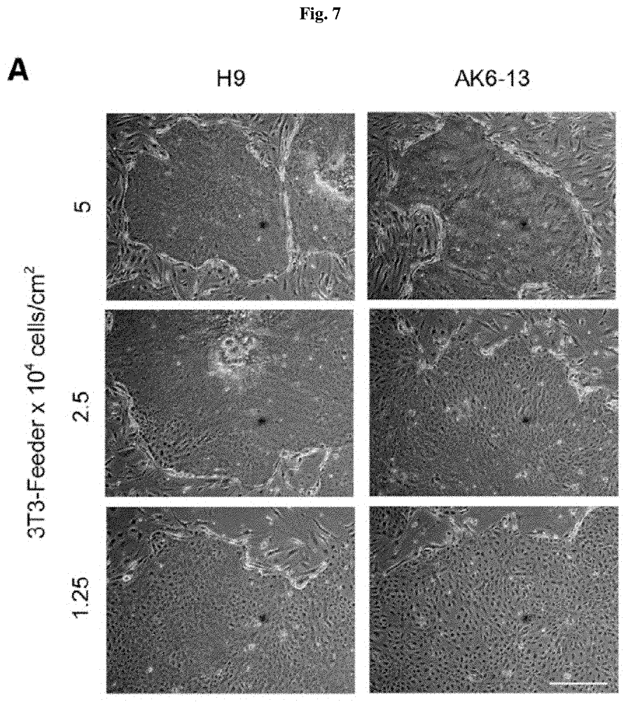

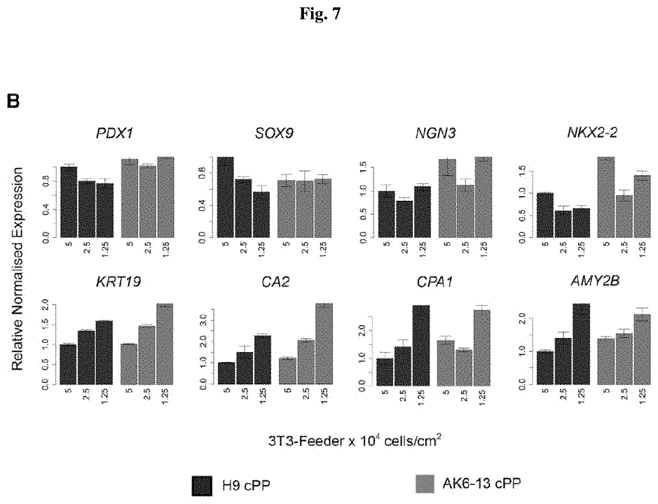

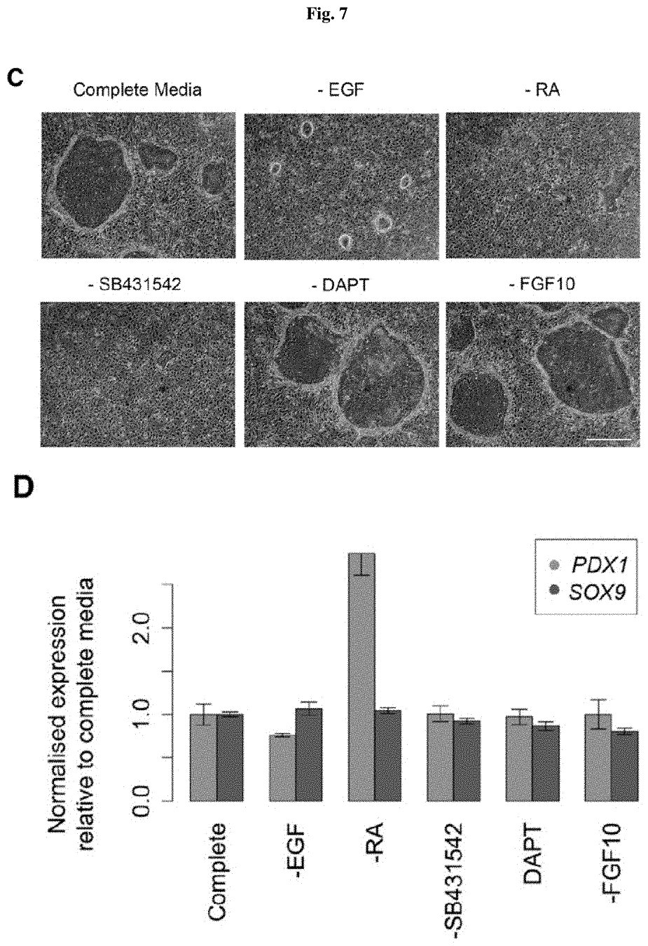

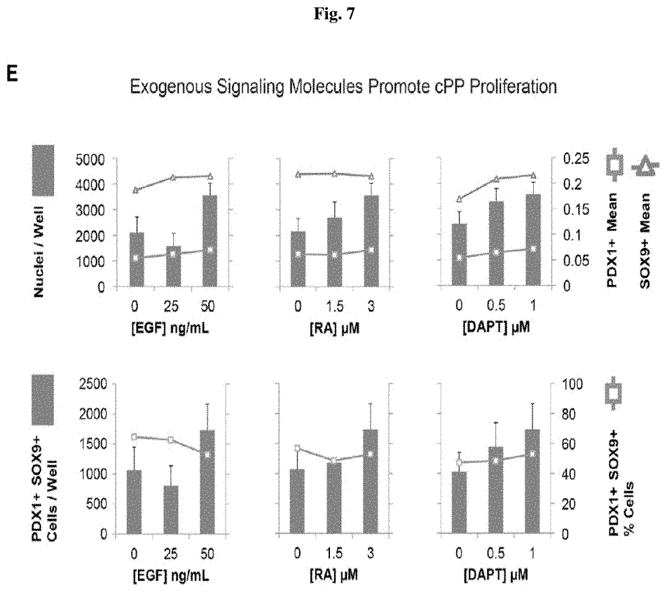

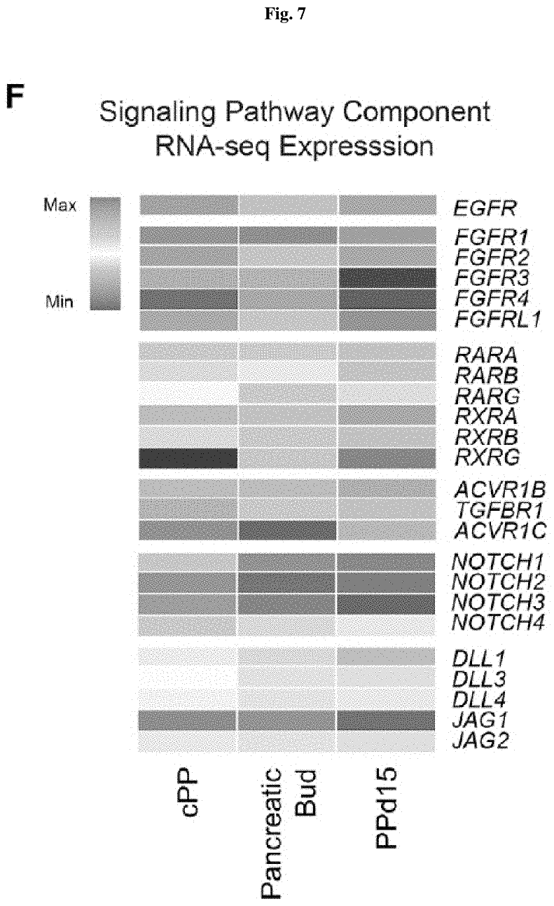

This study next investigated the roles played by the individual components of the culture system, specifically the layer of irradiated 3T3-J2 feeder cells, stimulation with EGF, FGF10, and retinoic acid (RA), and inhibition of the TGFβ and Notch signaling pathways. To assess the importance of the feeder layer, cPP cells were subcultured onto a layer of 3T3-J2 cells plated at decreasing densities and maintained in complete cPP culture media for 7 days. At reduced feeder densities, cPP cells continued to proliferate rapidly but quickly altered their morphology and could not be serially passaged (FIG. 7A). The levels of PDX1 and SOX9 remained stable, indicating cPP cells are committed to the pancreatic lineage, while markers of duct (KRT19 and CA2) and acinar (CPA1 and AMY2B) differentiation were upregulated (FIG. 7B). However, upregulation of endocrine markers (NGN3 and NKX2-2) was not observed, suggesting that 3T3-J2 feeder cells are required to block further differentiation toward the ductal and acinar linages.

To establish the roles played by the growth factors and small molecules in our culture media, each was removed individually and the effect on differentiation and proliferation was assessed. Exclusion of EGF or RA prevented cPP expansion, while removal of the TGF-β inhibitor SB431542 caused colonies to detach from the feeder layer (FIG. 7C). Removal of either FGF10 or the g-secretase inhibitor DAPT did not significantly affect colony size or morphology in the short term but, when removed from the culture media over multiple passages, led to a noticeable loss of viability. Interestingly, none of the growth factors or signaling inhibitors was individually required for maintenance of PDX1 or SOX9 expression (FIG. 7D). Indeed, removal of RA actually increased PDX1 expression. These results suggest that the growth factors and inhibitors present in our culture media are primarily required to drive proliferation of cPP cells rather than maintain their developmental state.

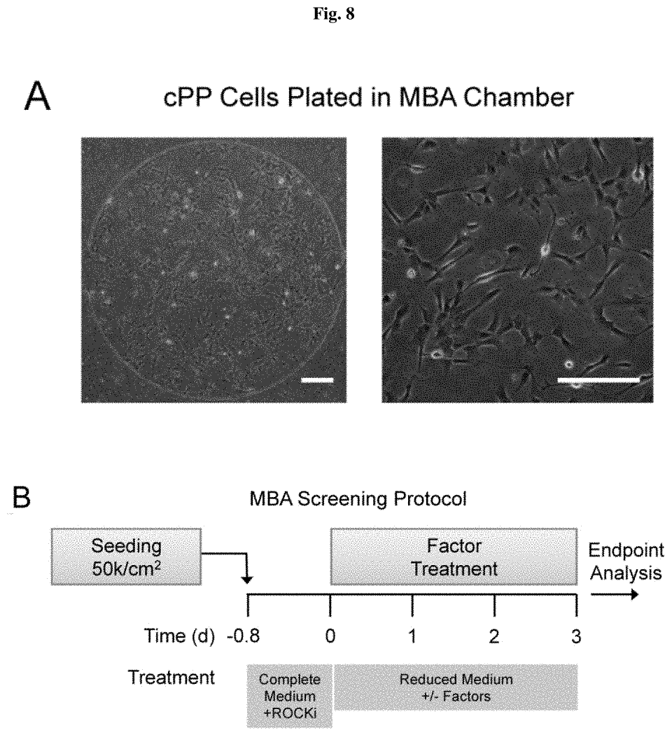

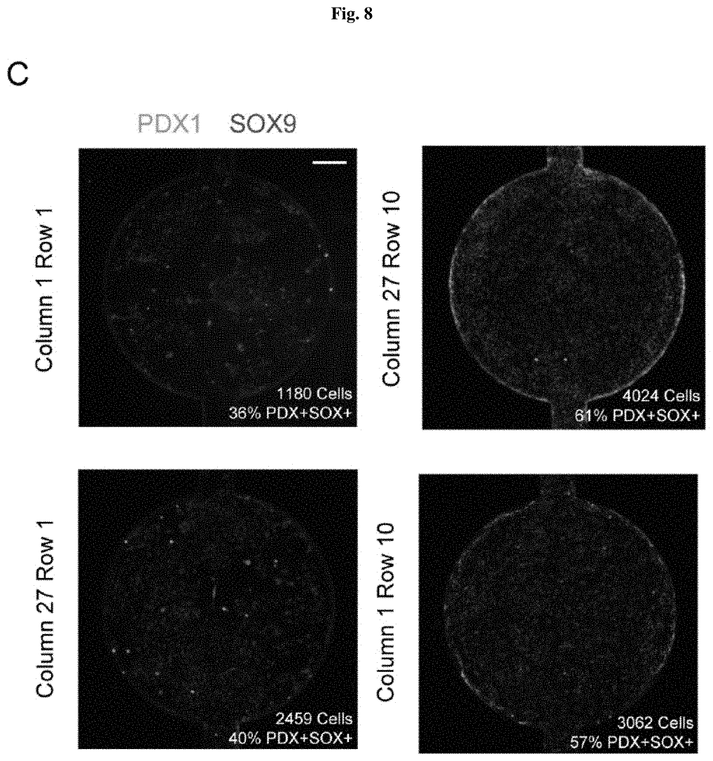

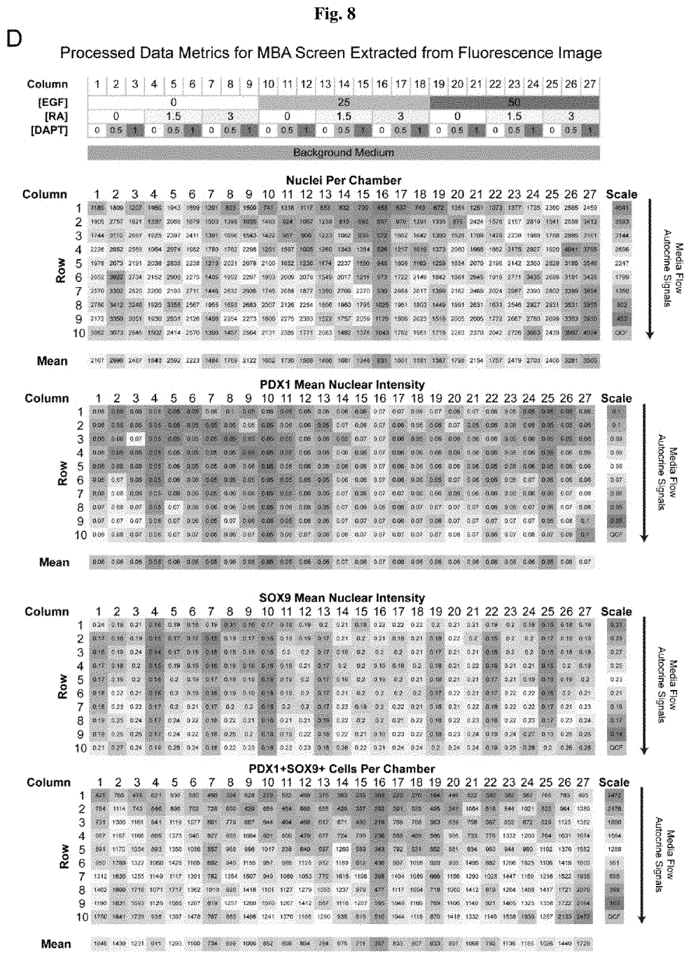

To quantify the effect of exogenous signaling molecules on the maintenance and expansion of cPP cells, this study used a microbioreactor array (MBA) screening platform to measure differentiation and proliferation. Single cPP cells were seeded in Matrigel-coated culture chambers in the absence of feeders and exposed for 3 days to complete cPP culture media in which the levels of EGF, RA, and DAPT were varied (FIG. 8). The study then used an image-segmentation algorithm to identify individual nuclei and quantify immunofluorescence staining for PDX1 and SOX9, thereby enabling the determination of the percentage of double-positive cells following exposure to different growth factor regimes. Reducing the levels of any of the three factors led to a reduction in both the total number of cells and the number of PDX1+ SOX9+cells (FIG. 7E). However, neither the mean levels of PDX1/SOX9 nor the percentage of PDX1+ SOX9+cells were dependent on the levels of these factors, suggesting they act primarily as mitogens. Interestingly, an increase in the number and percentage of PDX1+SOX9+ cells was noticed, but no change in the overall proliferation rate, when cells were exposed to higher concentrations of autocrine signals, particularly when provided with maximal levels of EGF, RA, and DAPT (FIG. 8D). Exposure to endogenous soluble signaling molecules is therefore required to maintain PDX1 and SOX9 independently of proliferation.

When taken together, these observations demonstrate that self-renewal of cPP cells is dependent on activation of the EGF, FGF10, and RA pathways and inhibition of Notch signaling. Indeed, cPP cells and their in vitro (PPd15) and in vivo (CS16-18 pancreatic progenitor) equivalents expressed high levels of multiple receptors of EGF, FGF, RA, and Notch signaling, as well as the TGFβ receptors ALK4 and ALKS (encoded by ACVR1 B and TGFBR1, respectively) that are inhibited by SB431542 (FIG. 7F). Consistent with the observations, production of FGF10 and RA by the surrounding mesenchyme is essential for expansion of the murine pancreatic bud, while EGFR is expressed throughout the pancreas and regulates islet development. Intracellular Notch signaling promotes expansion of pancreatic progenitors and prevents their further differentiation into endocrine cells. Therefore, this study's observation that the g-secretase inhibitor DAPT promotes proliferation of cPP cells is somewhat surprising. However, FGF10 has been shown to promote Notch activity in the developing pancreatic epithelium, and cPP cells express intermediate levels of the Notch effector HES1 relative to the 23 tissues described in FIG. 6A (data not shown). Therefore, the relatively low concentration of DAPT added to cPP cultures most likely serves to temper Notch activity, and exceptionally high levels of Notch activity might actually suppress proliferation.

Differentiation of cPP Cells into Pancreatic Cell Types In Vitro and In Vivo

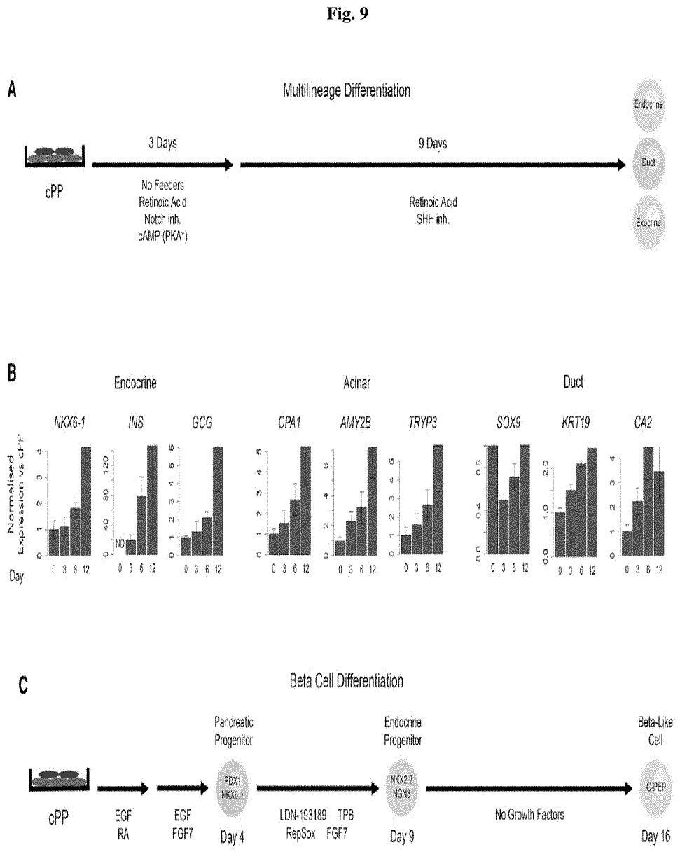

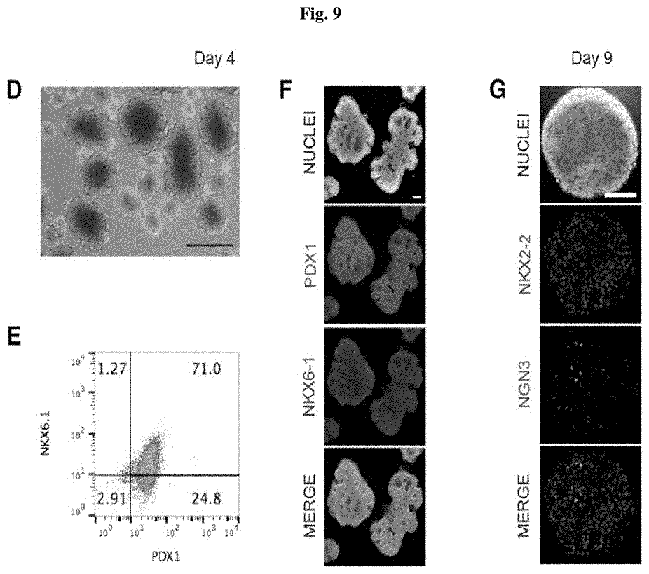

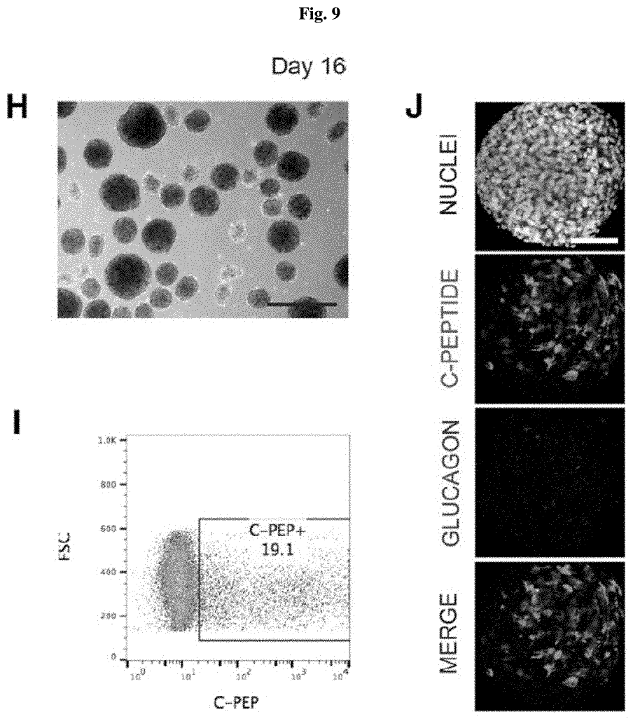

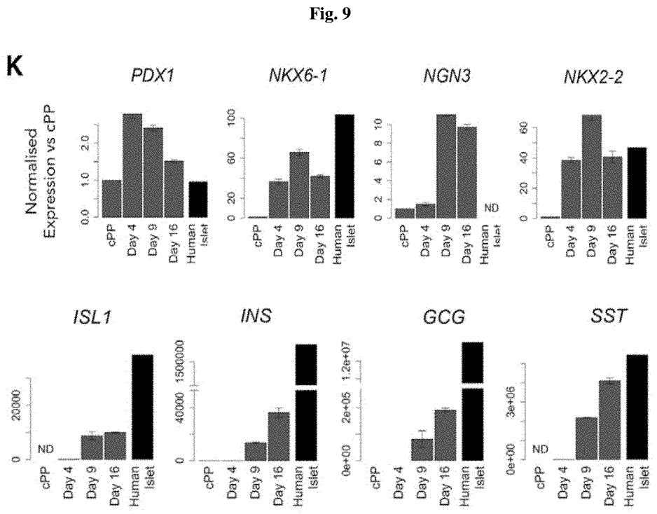

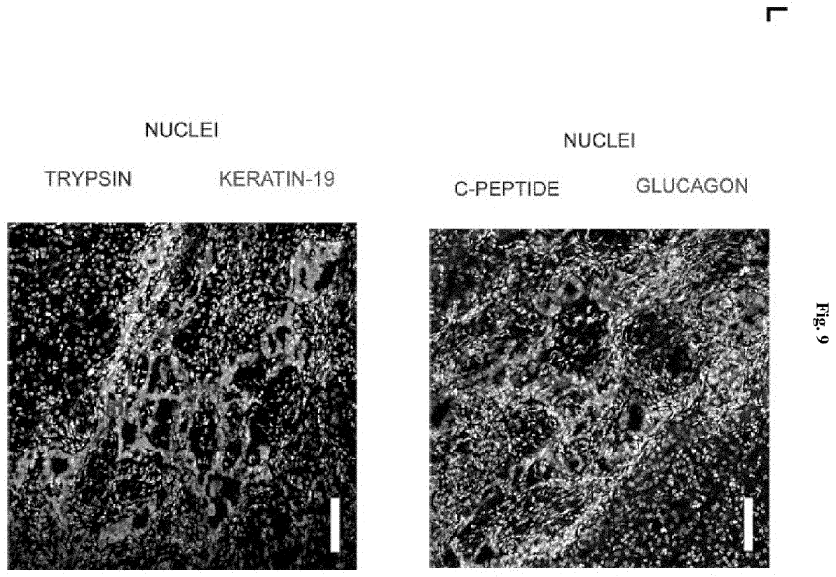

The canonical property of pancreatic progenitors is their ability to differentiate into each of the three lineages that constitute the pancreas as well as their functional derivatives. Initially, this study sought to determine whether cPP cells are capable of commitment to the endocrine, duct, and acinar lineages in vitro. Since robust protocols for the directed differentiation of pancreatic duct and acinar cells have yet to be developed, cPP cells were replated in the absence of feeders and exposed to a minimal signaling regime that promotes multilineage differentiation (FIG. 9A). Over the course of 12 days, upregulation of endocrine (NKX6-1, INS, and GCG), acinar (CPA1, AMY2B, and TRYP3), and duct (SOX9, KRT19, and CA2) markers was observed, demonstrating that cPP cells retain multilineage potency in vitro (FIG. 9B).

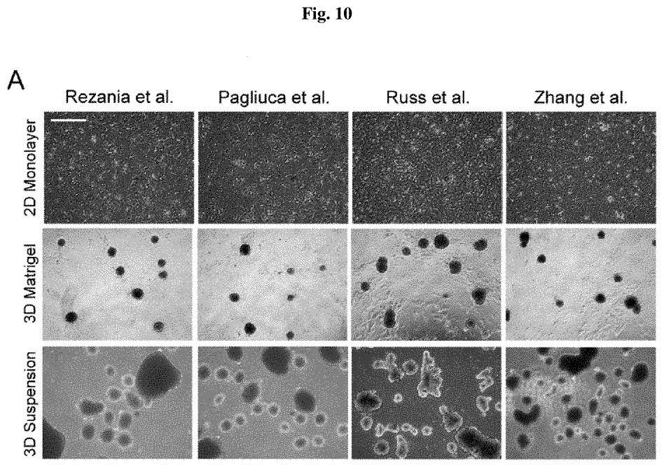

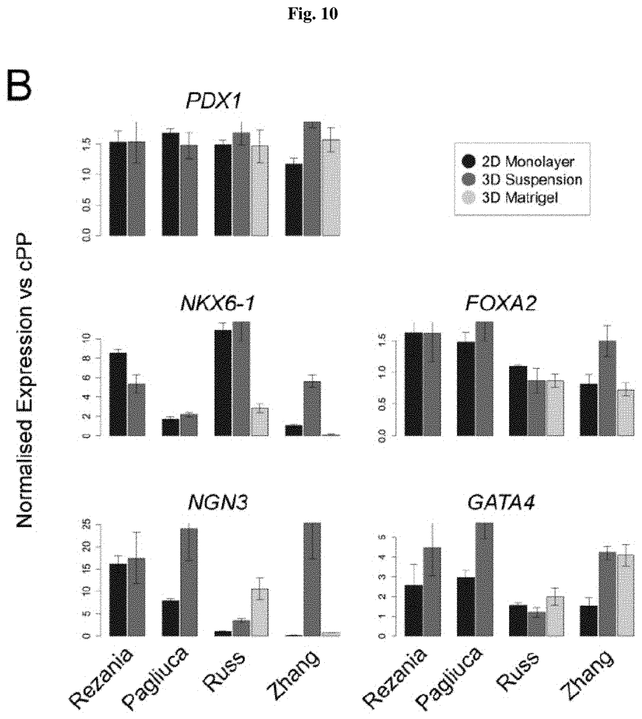

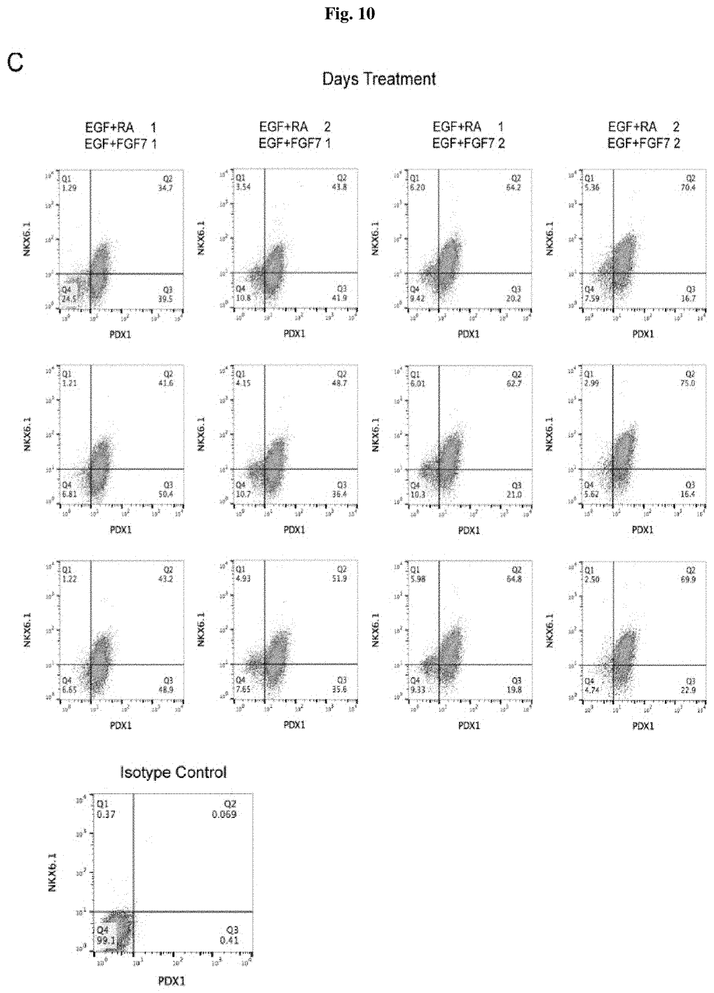

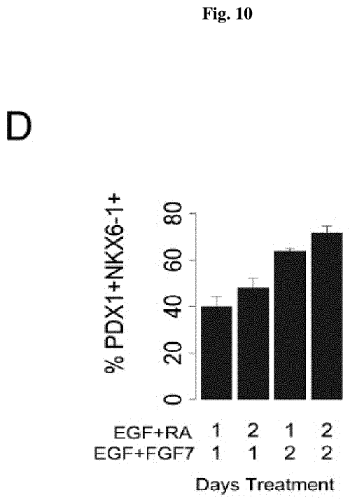

Of particular interest is the ability to generate b-like cells capable of secreting insulin in response to elevated glucose levels. Several groups recently published protocols that describe the differentiation of particular hESC and hiPSC cell lines into b-like cells. Activation of NKX6-1 prior to expression ofNGN3is thought to be essential for the formation of mature, functional β cells. Therefore, the four most promising protocols (Pagliuca et al., 2014; Rezania et al., 2014; Russ et al., 2015; Zhang et al., 2009) were selected and assessed their ability to induce NKX6-1 expression while maintaining low levels of NGN3. Specifically, cPP cells were cultured as monolayers or aggregates, then exposed to the section of each differentiation protocol shown to induce NKX6-1 expression (FIG. 10A). The protocol described by Russ et al. (2015) produced the highest levels of NKX6-1 expression and minimal activation of NGN3, with monolayer and suspension cultures yielding a very similar response (FIG. 10B). Since the original protocol demonstrated the generation of insulin-secreting b-like cells when cells were differentiated as aggregates, this study chose to use the 3D suspension platform for subsequent experiments. Using the Russ et al. (2015) protocol, the study found that around 40% of cPP cells reactivate NKX6-1. However, doubling the length of each of the first two treatments enabled the generation of nearly 70% double-positive cells, similar to the number originally reported (FIG. 9E, 10C, and 10D). Interestingly, these PDX1+NKX6-1+ cells generated convoluted structures reminiscent of the branching morphogenesis of the embryonic pancreas (FIG. 9D and 9F). Further differentiation induced expression of the endocrine markers NKX2-2 and NGN3, the latter in a smaller subset of cells, reflecting its transient expression during endocrine commitment (FIG. 9G). Finally, after 16 days, 20% of cells contained C-peptide, a proxy for insulin production, similar to the 25% reported by Russ et al. (2015). Crucially, C-peptide+ cells did not co-express the a cell hormone glucagon, suggesting that these cells are unlike the polyhormonal cells produced by earlier generations of protocols, which are unable to secrete insulin in response to elevated glucose levels. However, NGN3 levels remained high at the end of the protocol and INS mRNA levels were significantly lower than in isolated human islets, suggesting that further optimization of the protocol is required (FIG. 9K).

The most stringent test of developmental potency is whether a progenitor can differentiate into a particular lineage in vivo. To assess the potency of cPP cells, these cells were injected under the renal capsules of immunodeficient mice and immunostained for markers of the three major pancreatic lineages after >23 weeks. Large areas of cells expressing the b-cell marker C-peptide were able to be identified as well as the duct marker keratin 19 (KRT19), but this study was unable to find trypsin+ acinar cells or glucagon+ endocrine cells (FIG. 9L). However, trypsin+ cells were also observed rarely in prior studies following transplantation of pancreatic progenitors, possibly because acinar cells cannot survive in the absence of ducts to carry away the digestive enzymes they secrete. The absence of cells expressing glucagon was surprising, but likely reflects generation of C-peptide+ cells by default in the absence of inductive signals required to form glucagon+a cells.

The C-peptide+ cells did not form classical islet-like structures, but instead formed a series of interconnected cystic structures, as others have observed previously. Furthermore, this study did not observe expansion of the progenitor population once transplanted, suggesting cPP cells differentiate rapidly into less proliferative cells in vivo. Accordingly, none of the 12 mice assessed exhibited teratoma formation, despite transplanting >3 million cells into each mouse. These observations demonstrate that cPP cells retain the ability to differentiate into endocrine and duct cells in vivo, although it remains to be seen whether they are capable of forming acinar cells. Furthermore, the absence of teratoma formation suggests cPP cells may represent a safer alternative for transplantations than cells differentiated directly from pluripotent stem cells.

### DISCUSSION

Pluripotent stem cells have been proposed as an unlimited source of β cells for modeling and treating diabetes. However, the routine generation of functional β cells from diverse patient-derived hiPSC remains a challenge, partly because of the variability inherent in long, multi-step directed differentiation protocols. This study describes a platform for long-term culture of self-renewing pancreatic progenitor cells derived from human pluripotent stem cells. These cPP cells are capable of rapid and prolonged expansion, thereby offering a convenient alternative source of β cells. Furthermore, cPP cells can be stored and transported as frozen stocks, and cPP cells have been cultured for at least 25 passages with no loss of proliferation. It was observed that cPP cells express markers of pancreatic endocrine, duct, and acinar cells when differentiated in vitro, thereby demonstrating their multipotency, and this study was able to generate up to ˜20% C-peptide+ cells using a modified version of the βcell differentiation protocol described by Russ et al. (2015). The definitive test of developmental potency is whether a cell can differentiate into a particular lineage in vivo, and cPP cells indeed generate significant numbers of keratin-19+ duct cells and C-peptide+b-like

cells when transplanted under the renal capsule of an immunodeficient mouse, although it is unclear whether they retain the ability to form acinar cells in vivo.

Cells differentiating in vitro typically do so in an unsynchronized manner, causing cultures to become progressively more heterochronic with time and reducing the efficiency with which cells can be directed toward particular lineages. Therefore, the ability to capture and synchronize differentiating progenitors is essential for developing robust protocols for generating functional β cells from diverse genetic backgrounds. Extensive molecular characterization revealed that cPP cultures generated from both hESC and hiPSC represent stable populations of cells that express early pancreatic transcription factors consistently over time. The cPP transcriptome is closely related to that of the progenitor cells of the CS16-18 pancreas. However, comparison with human embryos at different stages of development suggests that cPP cells most closely resemble cells of the pancreatic bud between CS12 and CS13, based on robust expression of PDX1, SOX9, FOXA2, and GATA⁴/₆ and the absence of NKX6-1 and SOX17.

In recent years, several groups reported methods for culturing human endodermal derivatives. Two separate reports demonstrated that hESC-derived definitive endoderm can be serially passaged and expanded if cultured on a feeder layer in the presence of appropriate mitogenic signals. Subsequently, another group showed that foregut progenitor cells can be cultured in feeder-free conditions. However, slow growth and variable gene expression between different lines have limited their utility. More recently, it was shown that pancreatic progenitors derived from reprogrammed endodermal cells could be expanded and passaged. However, these cultures are highly heterogeneous, and it is not clear whether the minimal combination of signaling molecules and inhibitors used is sufficient to culture cells from different genetic backgrounds. Therefore, the culture system described here is the first to enable long-term self-renewal of multipotent pancreatic progenitors derived from genetically diverse hESC and hiPSC.

### REFERENCES

1. Russ, H. A., Parent, A.V., Ringler, J. J., Hennings, T. G., Nair, G. G., Shveygert, M., Guo, T., Puri, S., Haataja, L., Cirulli, V., et al. (2015). Controlled induction of human pancreatic progenitors produces functional beta-like cells in vitro. EMBO J. 34, 1759-1772.
2. Pagliuca, F. W., Millman, J. R., Gürtler, M., Segel, M., Van Dervort, A., Ryu, J. H., Peterson, Q. P., Greiner, D., and Melton, D. A. (2014). Generation of functional human pancreatic β cells in vitro. Cell 159, 428-439.
3. Rezania, A., Bruin, J. E., Arora, P., Rubin, A., Batushansky, I., Asadi, A., O'Dwyer, S., Quiskamp, N., Mojibian, M., Albrecht, T., et al. (2014). Reversal of diabetes with insulin-producing cells derived in vitro from human pluripotent stem cells. Nat. Biotechnol. 32, 1121-1133.
4. Zhang, D., Jiang,W., Liu, M., Sui, X., Yin, X., Chen, S., Shi, Y., and Deng, H. (2009). Highly efficient differentiation of human ES cells and iPS cells into mature pancreatic insulin-producing cells. Cell Res. 19, 429-438.
5. Micallef, S. J., Li, X., Schiesser, J. V., Hirst, C. E., Yu, Q. C., Lim, S. M., Nostro, M. C., Elliott, D. A., Sarangi, F., Harrison, L. C., Keller, G., Elefanty, A.G., Stanley, E. G., 2011. INS GFP/w human embryonic stem cells facilitate isolation of in vitro derived insulin-producing cells. Diabetologia 55,694-706.

1. A method of culturing a pancreatic progenitor cell comprising contacting said cell with:
a. epidermal growth factor (EGF);
b. retinoic acid (RA);
c. an inhibitor of transforming growth factor-β (TGF-β) signaling; and
d. 3T3-J2 fibroblast feeder cells.

2. The method of claim 1, wherein the inhibitor of transforming growth factor-β (TGF-β) signaling is an inhibitor of activin receptor-like kinase (ALK) receptor, optionally wherein the inhibitor of activin receptor-like kinase (ALK) receptor is SB431542.

3. (canceled)

4. The method of claim 1, wherein the pancreatic progenitor cell is further contacted with B27 supplement.

5. The method of claim 1, wherein the pancreatic progenitor cell is further contacted with an inhibitor of Notch signaling, optionally wherein the inhibitor of Notch signaling is a v-secretase inhibitor, optionally wherein the v-secretase inhibitor is DAPT.

6. and 7. (canceled)

8. The method of claim 1, wherein the pancreatic progenitor cell is further contacted with dexamethasone, fibroblast growth factor 10 (FGF10), N2 supplement or combinations thereof.

9. The method of claim 1, wherein the pancreatic progenitor cell is contacted with:
a. about 1 ng/ml to about 100 ng/ml of EGF;
b. about 100 nM to about 10μM of RA; and
c. about 1 μM to about 100μM of SB431542.

10. The method of claim 1, wherein the pancreatic progenitor cell is contacted with:
about 1 ng/ml to about 100 ng/ml of EGF;
about 1 ng/ml to about 100 ng/ml of FGF10;
about 100 nM to about 10μM of RA;
about 1 nM to about 100 nM of dexamethasone;
about 100 nM to about 10μM DAPT;
about 1 μM to about 100μM of SB431542;
about 1× B27 supplement; and
about 1× N2 supplement.

11. The method of claim 5, wherein the pancreatic progenitor cell is contacted with:
about 50 ng/mL EGF;
about 50 ng/ml FGF10;
about 3 μM RA;
about 30 nM dexamethasone;
about 1 μM DAPT;
about 10 μM SB431542;
about 1× B27 supplement; and
about 1× N2 supplement.

12. The method of claim 1, wherein the pancreatic progenitor cell is a pancreatic progenitor cell population.

13. The method of claim 1, wherein the pancreatic progenitor cell population is substantially homogenous.

14. The method of claim 1, wherein the pancreatic progenitor cell population is at least 60%homogenous.

15. The method of claim 14, wherein the pancreatic progenitor cell population is at least 99% homogenous.

16. The method of claim 1, wherein the pancreatic progenitor cell is cultured for at least 5 passages, at least 10 passages, at least 15 passages, or at least 20 passages.

17. The method of claim 1, wherein the pancreatic progenitor cell is derived from a stem cell, optionally wherein the stem cell is a human embryonic stem cell (hESC), optionally wherein the stem cell is an induced pluripotent stem cell (iPSC).

18. and 19. (canceled)

20. The method of 19 claim 1, wherein the pancreatic progenitor cell expresses PDX1, SOX9, HNF6, FOXA2, and GATA6.

21. The method of claim 1, wherein the pancreatic progenitor cell does not express SOX2.

22. A cell produced according to the method of claim 1.

23. A kit when used in the method of claim 1, comprising one or more containers of cell culture medium, together with instructions for use.

24. The kit according to claim 23, wherein the kit further comprises 3T3-J2 feeder cells.


SEQUENCE LISTING (58 sequences)

SEQ ID NO: 1 - DNA, 20 bp, Artificial Sequence
  Primer sequence
atgccttctgacagagcact

SEQ ID NO: 2 - DNA, 20 bp, Artificial Sequence
  Primer sequence
acagcctagcatcccagaag

SEQ ID NO: 3 - DNA, 23 bp, Artificial Sequence
  Primer sequence
cagactccgaaggaagttgtatg

SEQ ID NO: 4 - DNA, 23 bp, Artificial Sequence
  Primer sequence
tttggggtggtgtaacaaatgat

SEQ ID NO: 5 - DNA, 20 bp, Artificial Sequence
  Primer sequence
gccaagtatgacccttccct

SEQ ID NO: 6 - DNA, 20 bp, Artificial Sequence
  Primer sequence
ccacgttgaaagcatgacca

SEQ ID NO: 7 - DNA, 20 bp, Artificial Sequence
  Primer sequence
ctgaccatcatggagcacac

SEQ ID NO: 8 - DNA, 20 bp, Artificial Sequence
  Primer sequence
gccagagaggaggacaagaa

SEQ ID NO: 9 - DNA, 23 bp, Artificial Sequence
  Primer sequence
atgacatcaagagctactttggc

SEQ ID NO: 10 - DNA, 21 bp, Artificial Sequence
  Primer sequence
ggcgaacagcaatcaggaact

SEQ ID NO: 11 - DNA, 18 bp, Artificial Sequence
  Primer sequence
gggagcggtgaagatgga

SEQ ID NO: 12 - DNA, 22 bp, Artificial Sequence
  Primer sequence
tcatgttgctcacggaggagta

SEQ ID NO: 13 - DNA, 20 bp, Artificial Sequence
  Primer sequence
tccctcttccctcctcaaat

SEQ ID NO: 14 - DNA, 20 bp, Artificial Sequence
  Primer sequence
tcagcgtgtaaaggcatctg

SEQ ID NO: 15 - DNA, 20 bp, Artificial Sequence
  Primer sequence
cagttcctacgcttcgcatc

SEQ ID NO: 16 - DNA, 20 bp, Artificial Sequence
  Primer sequence
ttggtcgaggtcagtgaaca

SEQ ID NO: 17 - DNA, 24 bp, Artificial Sequence
  Primer sequence
aagcatttactttgtggctggatt

SEQ ID NO: 18 - DNA, 25 bp, Artificial Sequence
  Primer sequence
tgatctggatttctcctctgtgtct

SEQ ID NO: 19 - DNA, 23 bp, Artificial Sequence
  Primer sequence
tcacagataccagcagcatcagt

SEQ ID NO: 20 - DNA, 19 bp, Artificial Sequence
  Primer sequence
gggcatcaccaggcttgta

SEQ ID NO: 21 - DNA, 21 bp, Artificial Sequence
  Primer sequence
catggccaagattgacaacct

SEQ ID NO: 22 - DNA, 22 bp, Artificial Sequence
  Primer sequence
ttcccatatgttcctgcatcag

SEQ ID NO: 23 - DNA, 17 bp, Artificial Sequence
  Primer sequence
caggaggcgcatccaca

SEQ ID NO: 24 - DNA, 21 bp, Artificial Sequence
  Primer sequence
aagaggccatcaagcagatca

SEQ ID NO: 25 - DNA, 20 bp, Artificial Sequence
  Primer sequence
aaacaggagctccagcaaaa

SEQ ID NO: 26 - DNA, 20 bp, Artificial Sequence
  Primer sequence
agctacaggacaggccaaga

SEQ ID NO: 27 - DNA, 19 bp, Artificial Sequence
  Primer sequence
aacggcgagctagaggtga

SEQ ID NO: 28 - DNA, 21 bp, Artificial Sequence
  Primer sequence
ggatggtcgtgtagtagtggc

SEQ ID NO: 29 - DNA, 24 bp, Artificial Sequence
  Primer sequence
gctcatcgctctctattcttttgc

SEQ ID NO: 30 - DNA, 21 bp, Artificial Sequence
  Primer sequence
ggttgaggcgtcatcctttct

SEQ ID NO: 31 - DNA, 21 bp, Artificial Sequence
  Primer sequence
gggacttggagcttgagtcct

SEQ ID NO: 32 - DNA, 20 bp, Artificial Sequence
  Primer sequence
ggccttcagtactccctgca

SEQ ID NO: 33 - DNA, 20 bp, Artificial Sequence
  Primer sequence
cacacgagacccactttttc

SEQ ID NO: 34 - DNA, 20 bp, Artificial Sequence
  Primer sequence
ccgccaagtattttgtttgt

SEQ ID NO: 35 - DNA, 22 bp, Artificial Sequence
  Primer sequence
gtgttgcctctatccttcccat

SEQ ID NO: 36 - DNA, 18 bp, Artificial Sequence
  Primer sequence
cgctccgcttagcagcat

SEQ ID NO: 37 - DNA, 21 bp, Artificial Sequence
  Primer sequence
cctgctgggatattagctcca

SEQ ID NO: 38 - DNA, 19 bp, Artificial Sequence
  Primer sequence
cagcggtaggtgtcgaagc

SEQ ID NO: 39 - DNA, 21 bp, Artificial Sequence
  Primer sequence
aagtctaccaaagctcacgcg

SEQ ID NO: 40 - DNA, 15 bp, Artificial Sequence
  Primer sequence
gtaggcgccgcctgc

SEQ ID NO: 41 - DNA, 21 bp, Artificial Sequence
  Primer sequence
tgagaagcaacccttgtcatc

SEQ ID NO: 42 - DNA, 23 bp, Artificial Sequence
  Primer sequence
tcatcaacagactgactgcattc

SEQ ID NO: 43 - DNA, 20 bp, Artificial Sequence
  Primer sequence
atggagacgtttgaccccac

SEQ ID NO: 44 - DNA, 21 bp, Artificial Sequence
  Primer sequence
cgtagttgagccagcgatagt

SEQ ID NO: 45 - DNA, 19 bp, Artificial Sequence
  Primer sequence
ccgctgaccaaggatcgag

SEQ ID NO: 46 - DNA, 20 bp, Artificial Sequence
  Primer sequence
agggaacgggtttggctttc

SEQ ID NO: 47 - DNA, 24 bp, Artificial Sequence
  Primer sequence
agcggatcaatacctgtctcagaa

SEQ ID NO: 48 - DNA, 24 bp, Artificial Sequence
  Primer sequence
gcataaagaatgcaccgtggtaag

SEQ ID NO: 49 - DNA, 20 bp, Artificial Sequence
  Primer sequence
gaacgcacatcaagacggag

SEQ ID NO: 50 - DNA, 20 bp, Artificial Sequence
  Primer sequence
agttctggtggtcggtgtag

SEQ ID NO: 51 - DNA, 20 bp, Artificial Sequence
  Primer sequence
ccccagactccgtcagtttc

SEQ ID NO: 52 - DNA, 19 bp, Artificial Sequence
  Primer sequence
tccgtctggttgggttcag

SEQ ID NO: 53 - DNA, 20 bp, Artificial Sequence
  Primer sequence
tataatcccaagcggtttgc

SEQ ID NO: 54 - DNA, 20 bp, Artificial Sequence
  Primer sequence
gcacaccattttcccagaac

SEQ ID NO: 55 - DNA, 19 bp, Artificial Sequence
  Primer sequence
aaatgcggcccctgtttct

SEQ ID NO: 56 - DNA, 19 bp, Artificial Sequence
  Primer sequence
cagtgcgtcttgaggagca

SEQ ID NO: 57 - DNA, 20 bp, Artificial Sequence
  Primer sequence
catcaatgcggccaagatca

SEQ ID NO: 58 - DNA, 20 bp, Artificial Sequence
  Primer sequence
ggaattgatgacggcaggtg